(19) United States
(12) Patent Application Publication
(10) Pub. No.: US 2021/0264716 A1
(43) Pub. Date: Aug. 26, 2021

(54) INDIVIDUAL DEVICE AND SYSTEM TO DISPENSE PHARMACEUTICAL AGENTS TO AN AUTHORIZED PATIENT

(71) Applicant: Opio Connect, Inc., Kalispell, MT (US)

(72) Inventors: Amber Olivia Norbeck, Kalispell, MT (US); Melinda Fuzesy, Kalispell, MT (US); Michael Pokorny, Kalispell, MT (US)

(21) Appl. No.: 17/184,489

(22) Filed: Feb. 24, 2021

Related U.S. Application Data
(60) Provisional application No. 62/980,937, filed on Feb. 24, 2020.
(60) Provisional application No. 63/058,257, filed on Jul. 29, 2020.

(51) Int. Cl. G07F 17/00; G07F 11/58; G16H 20/13; G06F 21/32

(52) U.S. Cl. CPC G07F 17/0092; G06F 21/32; G16H 20/13; G07F 11/58

(57) ABSTRACT

According to aspects of the present disclosure, systems, apparatuses, and methods are provided to provide an authorized patient to receive prescribed pharmaceutical agents. In some embodiments the device is a handheld pharmaceutical agent delivery device. In other embodiments the authorized patient interacts with a kiosk system to provide both telemedicine and appropriate treatment including a dose of a pharmaceutical agent or a pharmaceutical agent delivery device.

### BACKGROUND

### Technical Field

The present disclosure relates to a device to dispense pharmaceutical agents to an authorized end-user through a kiosk system capable of exchanging the device once the pharmaceutical agent has been consumed. The kiosk system may include infrared technology or the capability to refrigerate pharmaceutical agents.

### Description of the Related Art

In the United States, it is estimated that 130 people die every day, nearly 50,000 deaths per year, from overdoses on opioids. In 2017, an estimated 1.7 million people suffered from opioid use disorder (OUD) related to prescription opioids, and 652,000 people suffered from heroin use disorder.

The misuse of prescription opioids alone, not including illicit street opioids, costs the U.S. nearly $80 billion per year. The White House Council on Economic Advisers estimated that in 2015, the total economic cost of the opioid crisis in the U.S. was $504 billion. That equals $1.38 billion a day. Fatality costs comprise over 85 percent of total costs, highlighting the crucial role played by mortality risk valuations when assessing the costs of this epidemic.

The two primary medications used to treat OUD are methadone and buprenorphine. Both medications are opioids, but they have vastly different regulatory controls. Methadone treatment for addiction is tightly regulated by federal guidelines and must be performed at a certified Opioid Treatment Program (OTP). Methadone initiation requires observed dosing for at least six days per week. There are very specific regulations on the maximum take home doses which is dependent on the duration in the treatment program. Many times, OTPs are only open a few hours a day making accessibility difficult, and many states have a very limited number of OTPs. In short, the use of methadone to address the opioid crisis is of limited effectiveness.

### BRIEF SUMMARY

The present disclosure relates to a pharmaceutical delivery device to deliver a prescribed pharmaceutical agent, such as transmucosal films or strips to an authorized user. In particular, the disclosure relates to a handheld device or wearable device configured to deliver a pharmaceutical agent for the treatment of opioid use disorder. The delivery device has one or more biometric sensor to authenticate the user as an authorized patient to receive the pharmaceutical agent from the device. In some instances the delivery device has facial recognition for dose confirmation and to authenticate the user. In some instances the delivery device has infrared technology, at times paired with automated decisioning (AD), to aid in detection or confirmation of diversion behavior. In some instances the delivery device will take time-stamped videos, photographs, or other images for dose confirmation to improve adherence. In some instances the delivery device is water resistant and tamper resistant. The delivery device has a display to facilitate use of the delivery device by the authenticated patient. The delivery device can be reusable or disposable. In some instances the reusable device can be open and loaded with a tamper resistant cartridge containing doses of a pharmaceutical agent. In some instances the tamper resistant cartridge has a tamper sensor.

In addition, the present disclosure relates a specialized kiosk system to provide telemedicine for a user. The kiosk system provides medical care to the user including a pharmaceutical agent or a pharmaceutical agent delivery device such as cartridges with a pharmaceutical agent. The kiosk has a biometric sensor, or a plurality of biometric sensor, to authenticate the user as an authorized patient to use the kiosk system. In some embodiments the biometric sensor include an artificial intelligence to perform facial recognition or biometric identification and automated decisioning (AD) to determine pharmaceutical agent dosing or potential diversion behaviors in the users. In some instances the kiosk system has infrared technology, at times paired with AD, to aid in detection or confirmation of diversion behavior. In some embodiments the kiosk system is contained in a privacy booth which may have a door. In some embodiments the kiosk system can dock with a pharmaceutical agent delivery device. In some embodiments the kiosk system can exchange a pharmaceutical agent delivery device for the authenticated patient. In some embodiments the kiosk system provides a single dose or multiple doses of a pharmaceutical agent. In some instances the pharmaceutical agent can be a partial opioid agonist, such as buprenorphine or a pure opioid agonist, such as methadone, or an opioid antagonist, such as naloxone. In some instances, the dosage of the pharmaceutical agent is provided in a child-proof safety package. In some instances a secure cartridge is provided that can be exchanged with cartridges in the kiosk, where the secure cartridge contains a delivery device, or a single dose or multiple doses of a pharmaceutical agent. The access to replace the secure cartridge may be protected by biometric authorization. The secure cartridge will interlock with the dispensing mechanisms of the kiosk to securely dispense either the delivery device or the single dose of a pharmaceutical to an authorized user.

The present disclosure also relates to the method of using both the pharmaceutical agent delivery device and the specialized kiosk system. The method of using the delivery device includes activating the device with biometric sensors to authenticate the authorized patient. The authorized patient requests a dose of the pharmaceutical agent held in the delivery device according to a dosing frequency programmed into the delivery device based on a prescription from a health care provider. The method of using the kiosk system includes activating the kiosk with biometric sensors to authenticate the authorized patient. The telehealth kiosk provides both telemedicine to the authorized patient and pharmaceutical agents to the patient. The dispensing kiosk solely dispenses pharmaceutical agents in delivery devices or as single doses to established authorized patients. The pharmaceutical agent provided from the kiosk can be based on the results of the telemedicine interaction with a health care provider or the authenticated prescription for a pharmaceutical agent. In some instances the authenticated patient will receive a single dose of a pharmaceutical agent from the kiosk or a pharmaceutical agent delivery device. In some instances the kiosk is capable of exchanging a used pharmaceutical agent delivery device for a new pharmaceutical agent delivery device. In some instances the new delivery device will contain the same prescription details as the used delivery device. In some instances the new delivery device will have an adjusted prescription.

These and other aspects will be evident upon reference to the attached drawings and following detailed description.

### BRIEF DESCRIPTION OF THE FIGURES

In the figures, identical reference numbers identify similar elements or acts. The sizes and relative positions of elements in the figures are not necessarily drawn to scale. For example, the shapes of various elements and angles are not drawn to scale, and some of these elements are enlarged and positioned to improve figure legibility.

FIG. 1 shows a schematic perspective view of the personal delivery device according to the present disclosure.

FIG. 2A is a top view of the pharmaceutical agent delivery device of FIG. 1 according to the present disclosure.

FIG. 2B is an end view of the pharmaceutical agent delivery device of FIG. 1 according to the present disclosure.

FIG. 2C is a side view of the pharmaceutical agent delivery device of FIG. 1 according to the present disclosure.

FIG. 2D is a bottom, perspective view of the pharmaceutical agent delivery device of FIG. 1 according to the present disclosure.

FIG. 3A is a cross-section view of the pharmaceutical agent delivery device of FIG. 1 through line 3A-3A according to the present disclosure.

FIG. 3B is a detailed view of the cross-section of the pharmaceutical agent delivery device of FIG. 3A according to the present disclosure.

FIG. 4 is a perspective view of an embodiment of the pharmaceutical agent delivery device of FIG. 1 according to the present disclosure.

FIG. 5 is an exploded view of an embodiment of the pharmaceutical agent delivery device of FIG. 1 according to the present disclosure.

FIG. 6 is a perspective view of an alternate embodiment of the pharmaceutical agent delivery device of FIG. 1 according to the present disclosure.

FIG. 7A is a perspective view of an alternate embodiment of the pharmaceutical agent delivery device according to the present disclosure.

FIG. 7B is a front view of an alternate embodiment of the pharmaceutical agent delivery device of FIG. 7A according to the present disclosure.

FIG. 7C is a side view of an alternate embodiment of the pharmaceutical agent delivery device of FIG. 7A according to the present disclosure.

FIG. 8 is a perspective view of a kiosk system according to the present disclosure.

FIG. 9A is side view of the kiosk system inside a privacy booth according the present disclosure.

FIG. 9B is a perspective view of the kiosk system inside a privacy booth according to the present disclosure.

FIG. 10 is a front view of the kiosk system of FIG. 8 according to the present disclosure, indicating a cross-section plane A and a cross-section plane B.

FIG. 11 is a cross-section view of the kiosk system through line A of FIG. 10 according to the present disclosure.

FIG. 12 is a side view of the kiosk system according to the present disclosure, indicating a cross-section plane C.

FIG. 13 is a view of cross-section plane C of the kiosk system according to the present disclosure from FIG. 12.

FIG. 14 is a cross-section view of the kiosk system through line B of FIG. 10 according to the present disclosure.

FIGS. 15A-15G are a method of using an embodiment of the kiosk system according to the present disclosure.

FIGS. 16A-16G are an alternative method of using an embodiment of the kiosk system, according to the present disclosure.

FIG. 17 is a block diagram of a medical treatment system 1700, according to one embodiment.

FIGS. 18 and 19 are perspective views of an alternative embodiment of a pharmaceutical agent delivery device.

### DETAILED DESCRIPTION

Unless the context requires otherwise, throughout the specification and claims which follow, the word “comprise” and variations thereof, such as “comprises” and “comprising” are to be construed in an open, inclusive sense, that is, as “including but not limited to”.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents unless the context clearly dictates otherwise. It should also be noted that the term “or” is generally employed in its broadest sense, that is, as meaning “and/or” unless the context clearly dictates otherwise.

As used in this specification, the term “automated decisioning” (AD) is interchangeable with automated decision-making or automated decision.

Buprenorphine significantly reduces cravings, illicit opioid use, and mortality, as well as improves psychosocial outcomes in patients with opioid use disorder (OUD). Medication assisted treatment with opioid agonist therapy, such as buprenorphine, has been shown to decrease mortality by approximately 50% among persons with OUD. Medication-based treatment with buprenorphine has also been shown to decrease healthcare costs by $20,000 per patient annually, when adherence is 80% or greater. It is the ideal medication to begin in the emergency department or after opioid overdoses. Unfortunately, only 20% of people with OUD are in medication-based treatment.

The worsening opioid epidemic has led to an increase in prescribing of this medication, in 2012, there were 9 million prescriptions for SUBOXONE®, accounting for a $1.5 billion market share. Buprenorphine is highly effective when taken appropriately as prescribed, however, without any controls in place when prescribing a significant quantity of opioids to a person with OUD, the consequence has been a drastic increase in diversion and subsequent new buprenorphine addiction. SAMHSA (Substance Abuse and Mental Health Services Administration) reported that buprenorphine was the highest misused prescription opioid in 2018, accounting for 718,000 cases (28.3%). Rural areas with lack of treatment for OUD have increased rates of diversion of buprenorphine as well as higher rates of opioid overdose deaths.

### Pharmaceutical agent Delivery Device

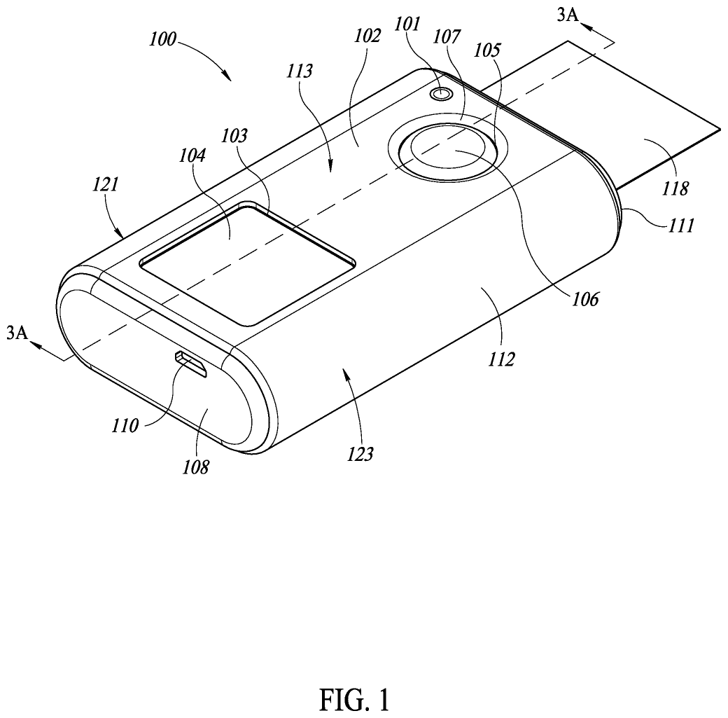

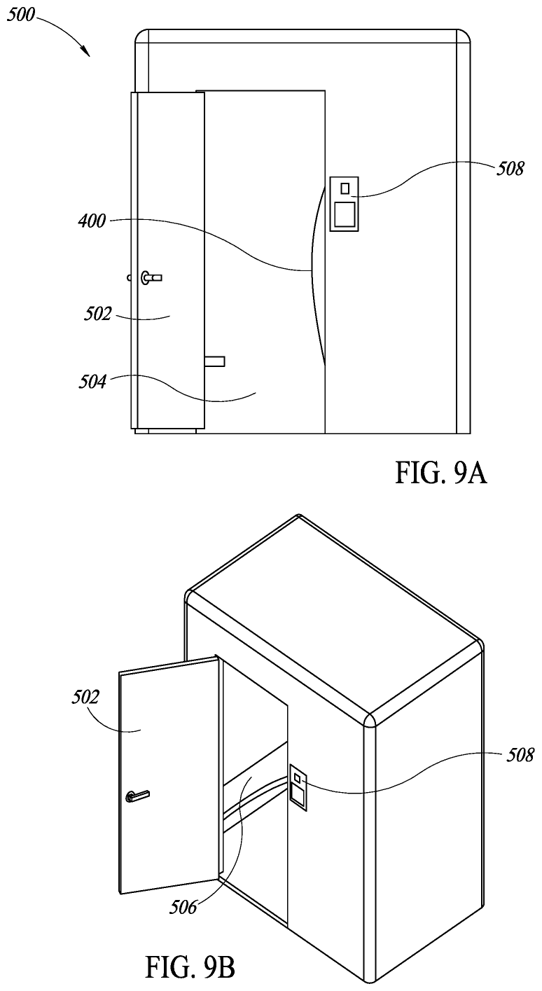

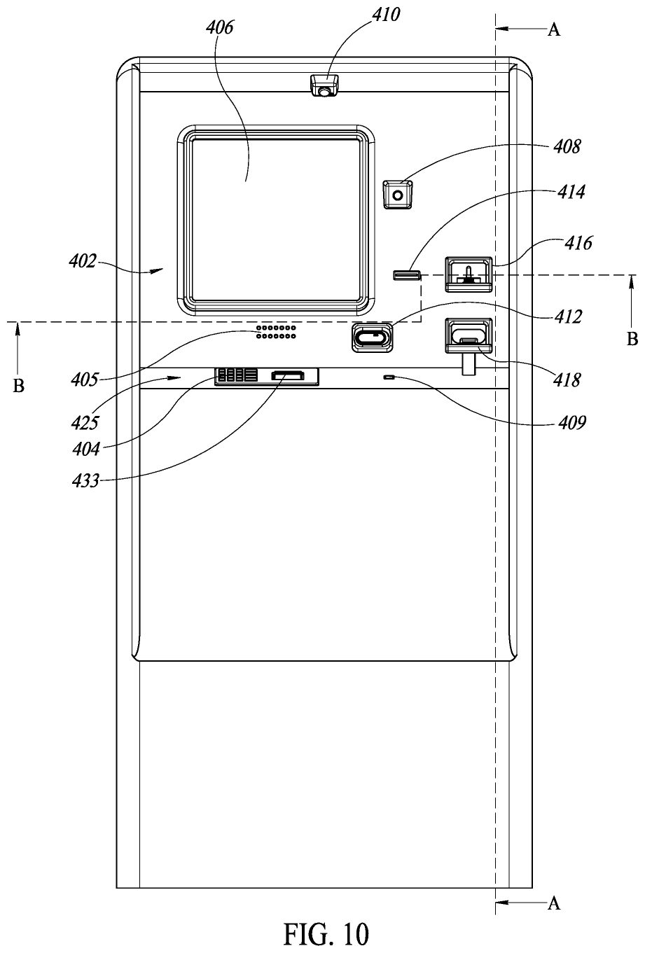

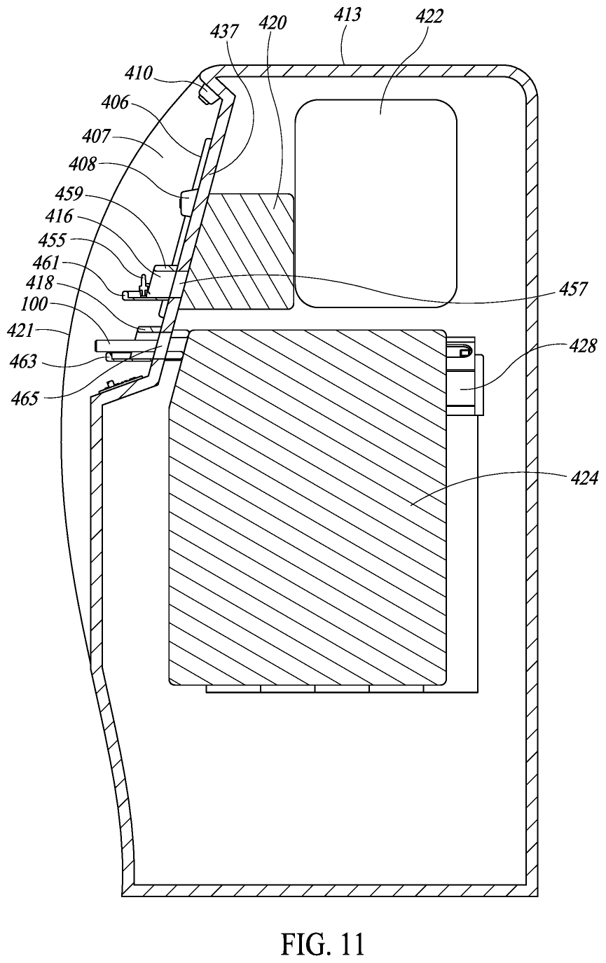

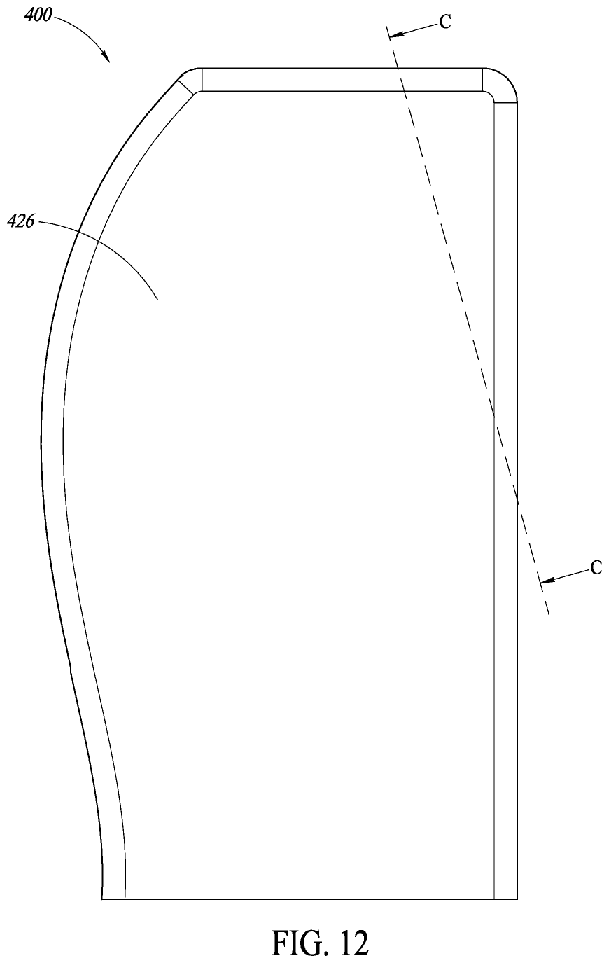

To address the issues with prescribing buprenorphine there needs to be an increase in the availability of treatment, including telemedicine to provide this medication in a secure environment with strict therapeutic monitoring. In addition, the use of biometric identification for the prescription of a controlled substances, such as buprenorphine, will lead to improved adherence and harm reduction through decreased mass diversion. Using a pharmaceutical agent delivery device, such as pharmaceutical agent delivery device 100 as depicted in FIG. 1, to dispense a pharmaceutical agent using biometric identification and improved security provides a novel method to address these problems. Additional embodiments of the pharmaceutical agent delivery device are depicted in FIGS. 6 and 7A-7C. In particular, a device capable of dispensing a single dose of a pharmaceutical agent, such as buprenorphine, to an authorized user adhering to proper prescription frequency and dosing will address the potential for abuse. This personal secure pharmaceutical agent delivery device will be capable of dispensing individual opioid films, strips, transmucosal products, or patches adhering to prescribed doses and frequency. The device may locate the nearest kiosk for a refill or exchange. The device can also interact via chat to order an exchange device through the specialty mail order pharmacy, or to the nearest pharmacy that is able to refill or exchange the device. The device may also access the software application and access telemedicine. It is possible that the device may essentially be a smart phone or smart watch that dispenses pharmaceutical films, strips or patches.

In some instances, the secure pharmaceutical agent delivery device will contain a processor to facilitate programing the device with specific dosages and frequency of the prescription. This could include a lock-out period in which the device will not dispense a dose even to an authorized patient. This delivery device would decrease the risks of abuse through the secure storage of the medication by decreasing misuse and ensuring proper dose, frequency, and duration of dispensing the pharmaceutical agent, such as buprenorphine, only to the authorized patient.

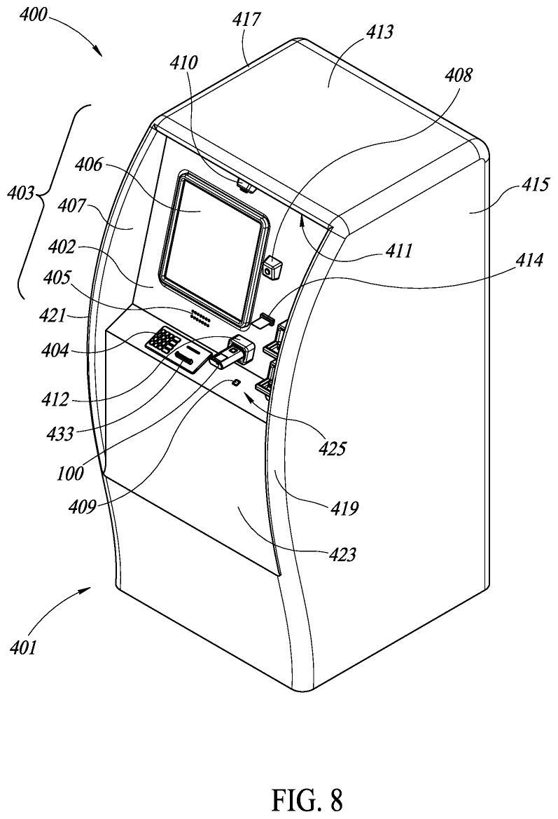

The device may include encrypted secure identifiable information connecting the authorized patient, the contents, the prescribed dose, frequency and duration. That is, the device will be configured to only dispense the correct medication to the intended user upon secure identification thereof and will not dispense to others. A plurality of various dispensing ranges, time intervals, or on-demand dispensing for cravings, as determined by the prescriber, are possible and are within the scope of this disclosure. Examples of identification strategies may include, but are not limited to, fingerprint identification, palm vein identification, palm print identification, face-recognition, retina identification, other biometric identification, multi-source identification software, and the like, such as fingerprint identification 206, and 306, or retinal identification 106, depicted in FIGS. 5-7A. Any identification means may be used herein.

The device may also include infrared sensors and/or video sensors 101 coupled with facial recognition to confirm pharmaceutical dose ingestion and to aid in the detection of any diversion behavior. Diversion behavior is the exchange, such as through selling, trading, loaning, or giving away, of controlled medications that results in the use of the controlled medications by individuals other than those for whom the controlled medication was prescribed. One example of a diversion behavior would be failing to ingest the pharmaceutical agent after it is dispensed in order to sell or trade it at a later time.

The device may contain norms established for the authorized user such that the facial recognition, microphone, video sensors, and infrared sensors may detect variations from these norms and alert health care providers of evidence of withdrawal, progression of abuse, adherence to program guidelines, or being under the influence. Norms may include pupillary dilation, eyelid position, clarity of speech, body temperature, levels of craniofacial erythema (facial flushing), grinding of teeth, etc. The device may also couple the data from the infrared sensors, video sensors, or facial recognition to automated decisioning (AD) software to aid in properly administration of the pharmaceutical agent or locking the device if diversion behavior is detected. The infrared system may monitor changes in the temperature of the pharmaceutical agent as it is being ingested by the patient to determine if the pharmaceutical agent is taken properly. The software analysis of any combination of information collected from infrared sensors and video sensors and shape tracking software may be used to track the medication to the patient's mouth to determine dose ingestion or diversion.

In some instances the AD software may improve the accuracy of identifying the authorized user. The AD may serve to detect over-sedation or impairment in the authorized user. The AD may serve to confirm dose ingestion by the authorized user which may also be time stamped, in some instances with photo or video confirmation. In some instances a time-stamped photograph or video is taken for dose confirmation to improve adherence. The AD may also serve to facilitate real-time alerts sent to the health care provider from the device and any real-time adjustments to the dosing of the pharmaceutical agent.

This delivery device may have a monitoring system within the device that provides feedback data to the prescribing provider to improve tracking and adherence to the prescription for long-term treatment regimens. An alert such as a vibration, visual alert on screen, or ringtone may notify the patient that the dose is due. The device may also check in with patient daily to get a daily craving score to assess risk for relapse and to provide positive feedback for adherence using chat bot or other technology. The patient may also message and ask questions using chatbot or other technology, this may include referrals to social services based on individual needs.

The delivery device that is used for securely housing and dispensing the pharmaceutical agent may include a tamper-resistant encasement. This tamper-resistant encasement may include an integrity detector which can trigger a substance within the device to render the pharmaceutical agent inert if the delivery device is tampered with.

The device may contain software that communicates with a loading device or docking station at the specialized kiosk or by an authorized prescriber. This software may communicate via wireless internet or via BLUETOOTH® to a smart phone. This loading system or docking station may include identification encryption methods to securely connect the contents to the identified authorized patient. The loading system or docking station is configured to securely lock and unlock the device for unloading and reloading. The secure locking and unlocking mechanism may include an electronic or magnetic system. Alternatively, the device may be loaded with a prefilled cartridge supplied by the manufacturer, the specialized kiosk, specialty mail order pharmacy, opioid treatment program (OTP), or elsewhere. This loading system may include identification encryption methods to securely associate the device, cartridge, and its contents to the identified user. The loading device or docking station may include a scanner, reader, or programing device to facilitate communication with the delivery device, to facilitate uploading data to the delivery device and from the dispensing device. This docking station may be located in the kiosk, pharmacy, or an OTP.

In some instances, a replaceable cartridge 134, as depicted in FIG. 5, that holds the medication may be used, in other instances, the cartridge may be omitted. The device will only be loaded with one opioid prescription medication (films, strips, transmucosal products, or patches) by authorized personnel or by a specialized kiosk configured specifically to exchange the dispensing device. The cartridge in the delivery device may hold a predefined number of the prescribed pharmaceutical agent units. The cartridge may be replaceable and inserted into the device. The replaceable cartridge may be tamper-resistant and only accessible by authorized personnel and or a specialized kiosk configured specifically to exchange the dispensing device. The delivery device may perform the same functions, with or without a cartridge, once loaded by the authorized personnel or the specialty kiosk. The device may be disposable, or it may be refillable and reusable.

The device may be configured to be hand held and carried in the pocket of a patient or it may be a wearable, such as a watch, pendant, or keychain. The device may include a processor, a memory, a power source, at least one power source, and a communication interface. The device will include a dispensing mechanism configured to dispense the pharmaceutical agent. This dispensing mechanism may include rolling or a pneumatic mechanism. The film, strip, transmucosal product, or patch, may be dispensed from one end of the device. The encasement of the delivery device will be highly durable to protect the medication, the replaceable cartridge, the processor, the memory, the power source, and the communication interface from attempts to tamper with the device. In some embodiments, the encasement is made of a durable material such as acrylonitrile butadiene styrene (ABS) plastic that is used in the aviation industry.

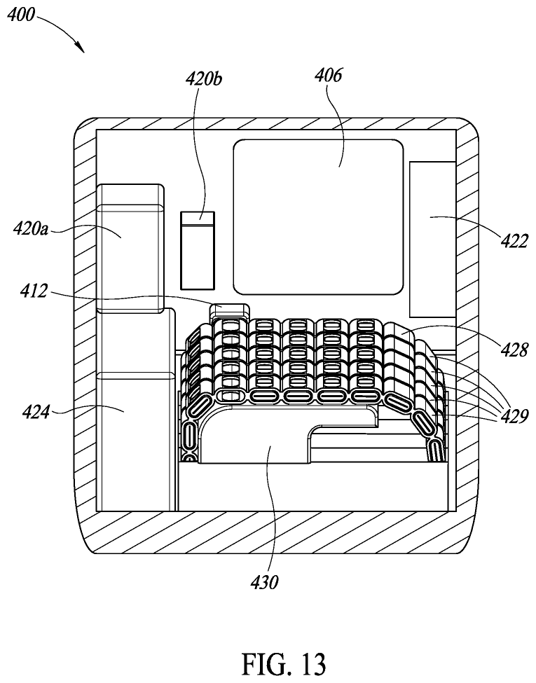

In some embodiments, the device may include an opening for inserting or removing the replaceable cartridge or the pharmaceutical agent. The cartridge may contain a plurality of individual doses of a pharmaceutical agent or a plurality of packages containing pharmaceutical agents. The opening will be resistant to tampering and will be accessible by authorized personnel or by a specialized kiosk made for the device, such as the kiosk system depicted in FIG. 8. The authorized personnel or specialized kiosk may insert or remove the replaceable cartridge or the pharmaceutical agent into or out of the dispensing device. In alternative embodiments, the opening will include a locking mechanism. In some instances, the locking mechanism may be an electronic lock, mechanical lock, biometric lock, or any combination of the foregoing. In some embodiments the person authorized to interact with the dispensing device, may be required to enter a code, provide a finger print, use a key to an electronic or magnetic mechanism, or any combination of the foregoing, to access the dispensing device. In accessing the delivery device the authorized person will be able to replace the pharmaceutical agent or cartridge containing the pharmaceutical agent and possibly any data contained in the dispensing device. Other locking mechanisms may also be used to prevent tampering with the device. The device may contain notification systems to alert the operating system directly or indirectly to tamper attempts or destruction of the device. In some instancing this integrity detector will trigger release of an agent to render any pharmaceutical agent in the delivery device inert.

In some embodiments, in order to securely dispense the pharmaceutical agent to the authorized patient, the patient will be required to provide biometric identification, non-limiting examples include: fingerprint, facial recognition, palm vein scan, palm print recognition, retina identification, and another form of encrypted identification, such as a personal identification number (PIN), to prevent unauthorized access to the prescribed substance by someone other than the patient. In some embodiments, a display screen may be used as a biometric sensor to collect patient biometrics to identify and authorize a patient. In some instances, patient registration, including biometric data, may be recorded at a secure telemedicine specialty kiosk or at the first visit at the authorized provider's office using software or a smart-phone application to interact with the device-specific software. Registration information may include patient demographics, prescription information (dose, frequency, etc.), biometric information (fingerprint, facial recognition, retina recognition, or the like, etc.) and any other necessary information needed to securely identify the authorized patient. The registration information may prevent any single person from getting multiple prescriptions for the same medication. This device-specific software may also interface with the controlled substances drug registry and provide a prescription history to the prescriber or pharmacist.

The device may include at least one power source (i.e., a battery), a processor in communication with at least one memory unit. The device may include wireless or cellular communication interface functionalities, such as BLUETOOTH®. The device may function independently or it may require access to Wi-Fi or a smart phone. The processor or memory may be configured to dispense the pharmaceutical agent from the delivery device as prescribed and may monitor how much drug has been dispensed, when, how many attempts, and may cease access if too many attempts to access medication occur. The delivery device may include a GPS module to track the delivery device if it is lost or stolen. There may be one or more sensors, such as infrared sensors, electromagnetic sensors, weight sensors, or cameras in the delivery device to improve the security and integrity of the dispensing device.

The device may include a remote monitoring component. The remote monitoring component may receive all data and activity that has occurred on the device. The device and remote monitoring component may interact with biosensors, capillary blood, or drug breathalyzers to test for opioid levels and other controlled substances. All data may be reportable to the prescriber for evaluation. The remote monitoring may have the capability to determine that the device is being tampered with and may send a signal to stop the dispensing of the medication altogether and may also destroy the contents of the medication within the device. This autodestruct functionality may be done with liquids, chemicals, heat, compression, or the addition of a counter substance to render the pharmaceutical agent inert.

Authorized personnel or a specialized kiosk for exchanging the device may reload the replaceable cartridge or refill the medication without a cartridge into the dispensing device. In some instances, the replaceable cartridge may be pre-loaded. The device may accommodate packaged films, strips, transmucosal products or patches; or may accommodate manufacturer ready-made unpackaged films, strips, transmucosal products or patches. The size of the device may change based on the size of the packaging of the medication as determined by the manufacturer.

In certain instances, when an electronic prescription is received, prescription information or patient information may be transmitted or entered into the database that interacts with the devices. Alternatively, authorized prescribers may use specific software designed to communicate with the dispensing devices. This may allow patient data, such as biometric data, demographics, the prescription information, and may include a mechanism to transmit and interface with loading and dispensing station at the specialized kiosk for the device or specialty pharmacy. As a non-limiting example, this delivery device could easily be distributed by an online pharmacy. The online pharmacy could ship the device fully programmed anywhere in the world. The specialty kiosk designed specifically to exchange used and new dispensing devices.

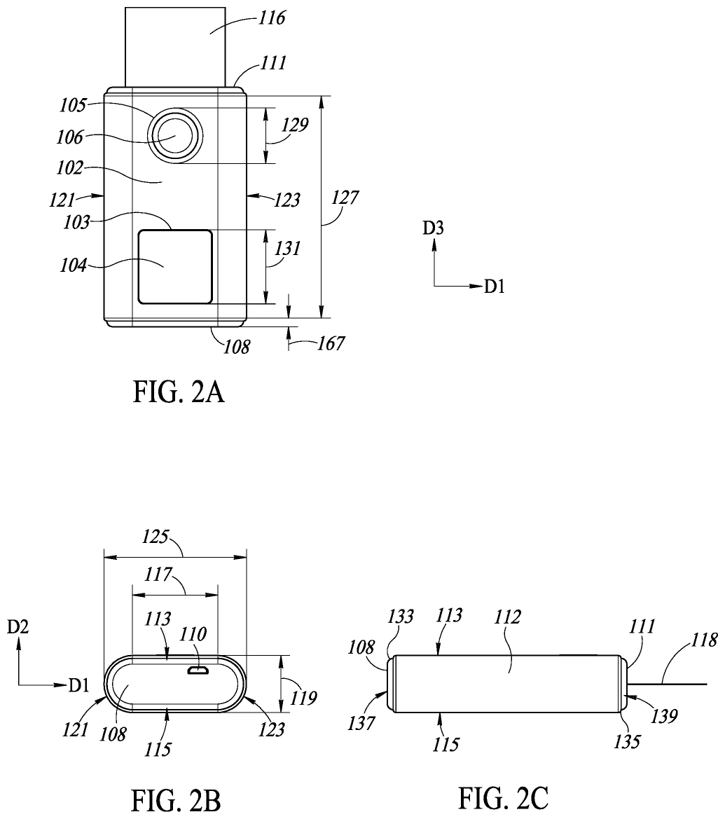

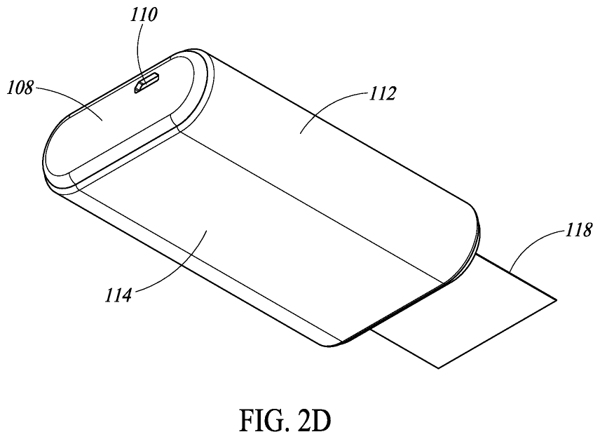

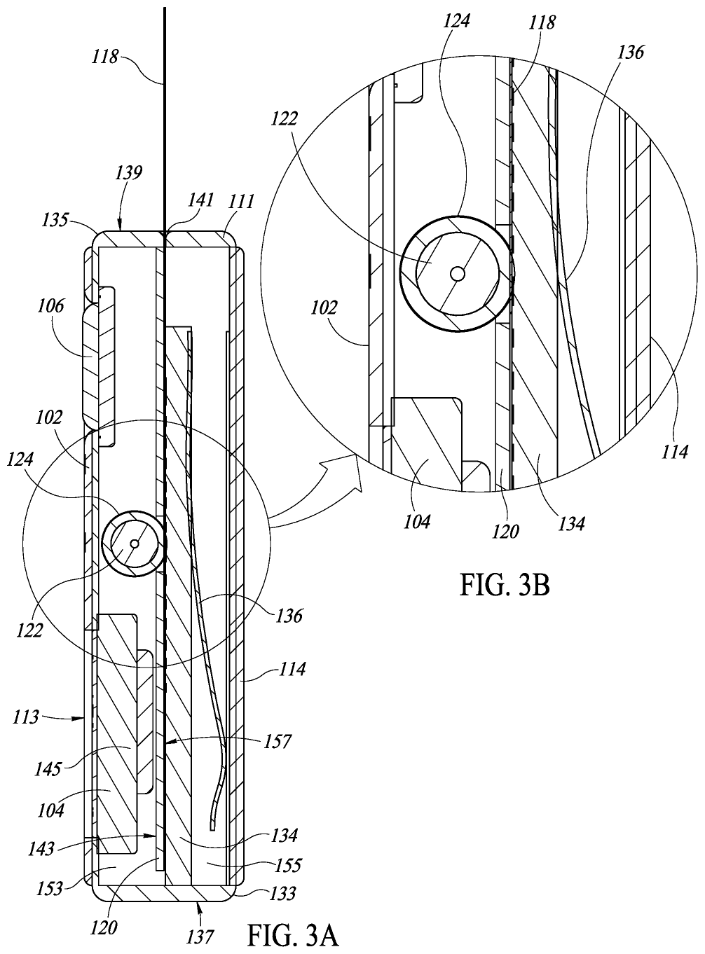

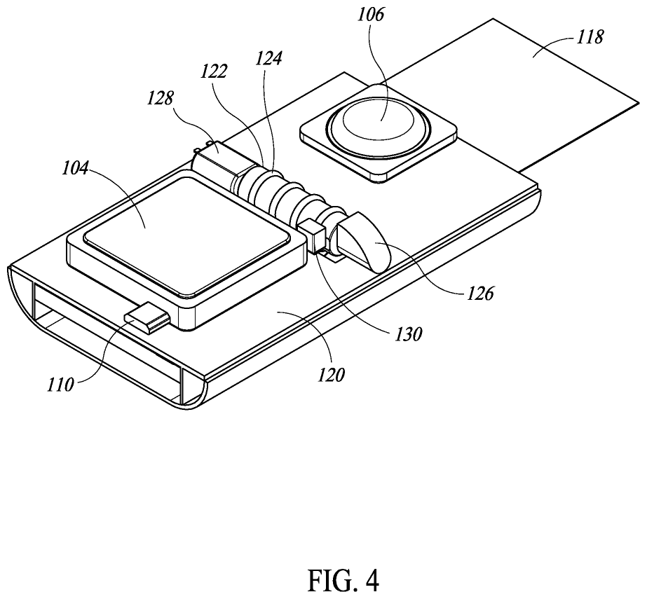

One aspect of the present disclosure is directed to a pharmaceutical agent delivery device 100 as depicted in FIGS. 1, 2A-2D, and 3-6. The delivery device 100 has a chassis that is includes an outer shell 112 with a first end 108 opposite a second end 111. The shell 112 of the chassis is disposed between the first end 108 and the second end 111. The shell 112 has a first side 102 opposite a second side 114 as seen in FIG. 2D. In some embodiments the outer shell 112 has a plurality of openings, including a first opening 105 and a second opening 103 on the first side 102. The first opening 105 is aligned with a biometric sensor 106 when the delivery device 100 is in a closed or sealed configuration.

The first opening 105 is circular in FIG. 1 and surrounds the biometric sensor 106. In some embodiments the biometric sensor 106 may be a retinal sensor that is supported on a substrate that is internal to the device 100. A portion of the sensor 106 will be housed within the chassis, with an interfacing portion extending through the first opening 105. The first opening 105 may have a different shape, such as square or rectangular, to accommodate different designs of biometric sensors. For example, in an alternative embodiment in FIG. 6, a delivery device 200 has a biometric sensor 206 that is recessed within a first opening 205. More details of FIG. 6 are included below.

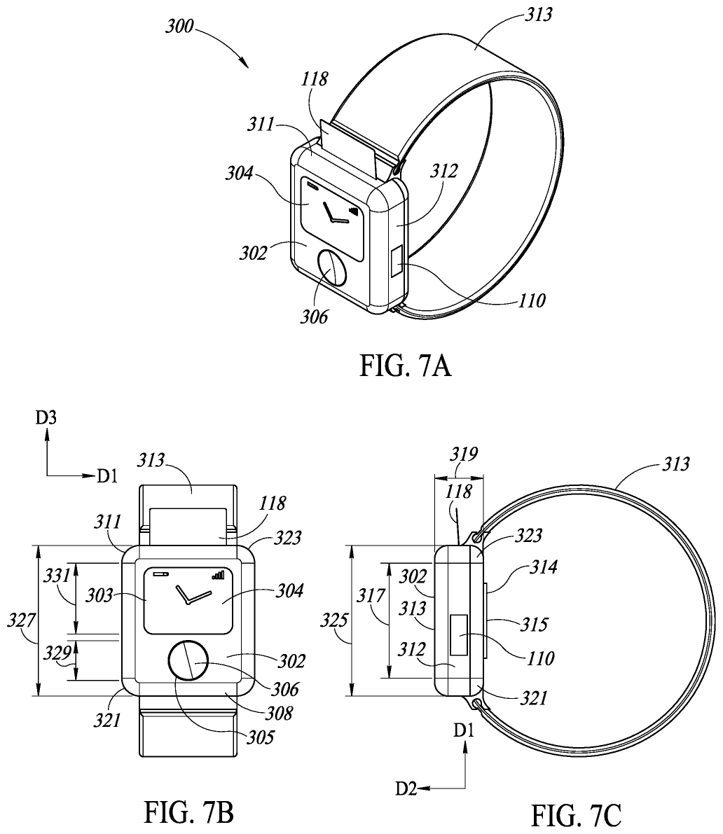

In FIG. 1, an optional camera 101 (which may be an image sensor or a video capture device) is positioned closer to the second end 111. The camera 101 is between the first opening and the second end 111. In some embodiments, the camera 101 may be aligned with the opening 105 along a center-line of the device. Alternatively, the camera may be positioned in a corner opposite to the currently illustrated corner. The camera includes a lens that is the external interface between the environment and the user and an internal chip or processor configured to capture, receive, and process images according the selected end use. The chip will be housed within the shell or housing 112. The chip will be mounted to or otherwise supported by a support substrate 120. The camera is not illustrated in FIGS. 2A-2D, 4 or others.

The first opening 105 of FIG. 1 includes a beveled or angled edge 107 that surrounds the biometric sensor and provide a transitioned edge for a user's comfort when using as a retinal sensor. The first opening is closer to the second end 111 than the second opening 103. The second opening 103 is aligned with a display 104 when the delivery device 100 is in a closed or sealed, ready for use configuration. The second opening 103 has a square or rectangular shape to provide sufficient surface area for the display. The display may be a touch screen or other electronic display to provide information and interact with the patient or user. For example, the display 104 may provide dosing, frequency, and biographical information of an authorized patient when the delivery device 100 is associated with the authorized patient. The display may provide information about battery life remaining and provide time and date information, including availability of the next dose according to the proscribed frequency. In some embodiments the pharmaceutical agent 118 is in the form of a film or strip. In some embodiments this film or strip can be 2.2 cm×1.3 cm which either unpackaged or individually packaged. In some embodiments the film or strip may be smaller than 2.2 cm×1.3 cm. In some embodiments the film or strip may be larger than 2.2 cm×1.3 cm.

The outer shell 112 is a single, hollow piece that includes the first and second openings 103, 105, see FIG. 5. The outer shell 112 has at least one open end 109 configured to accommodate an inner shelf or support substrate 120. The outer shell 112 includes a first flat surface 113 and a second flat surface 115, see FIG. 2B. The first and second flat surfaces 113, 115 have a first dimension 117 in a first direction D1. The first and second flat surfaces 113, 115 are spaced from each other by a second dimension 119 in a second direction D2.

The outer shell 112 includes a first curved surface 121 and a second curved surface 123. The curved surfaces are coupled to and integrally formed with the first and second flat surfaces 113, 115 in one embodiment. A third dimension 125 in the first direction, between an outermost point of the first and second curved surfaces is greater than the first dimension 117.

The outer shell includes a fourth dimension 127 between the first end 108 and the second end 111, See FIGS. 2A, 2B, and 2C. The fourth dimension 127 extends in a third direction that is transverse to the first and second directions D1, D2. The fourth dimension 127 is greater than the third dimension 125. A radius of the first opening 105 from the outermost edge of the bevel is a fifth dimension 129. The display in the second opening 103 includes a sixth dimension 131 that is greater than the fifth dimension 129. The fifth and sixth dimensions extend in the third direction D3.

The first end 108 has a dimension 167 in the third direction D3. This dimension 167 is a part of the first end that is exposed outside of the outer shell 112. A part (not shown) of the first end is positioned within the outer shell 112 when in a sealed, ready to distribute confirmation. The dimension 167 is less than the fifth dimension 129. The first and second ends are securely coupled within the outer shell 112, such as with a glue, internal latches, or security fasteners that can only be open with a manufacturer's specialized tool. For example, the fasteners could have a locking mechanism that only unlocks in response to a specific frequency or pattern of specific frequencies.

The first and second ends are physical components that couple to an interior surface of the outer shell 112 to securely and tightly seal the delivery device 100. The second end 111 of the delivery device 100 is configured to deliver a pharmaceutical agent 118. In some embodiments the pharmaceutical agent 118 is an individually wrapped medication, such as buprenorphine and buprenorphine/naloxone transmucosal film or strip, e.g., SUBOXONE®. The first end 108 of the delivery device 100 includes a data and charging port 110 that is operably linked to a processor or application specific integrated circuit and a battery. In some embodiments, the data port 110 may be a USB-C port. The data port 110 enables an authorized user to download or upload data to and from the delivery device 100 using encryption techniques.

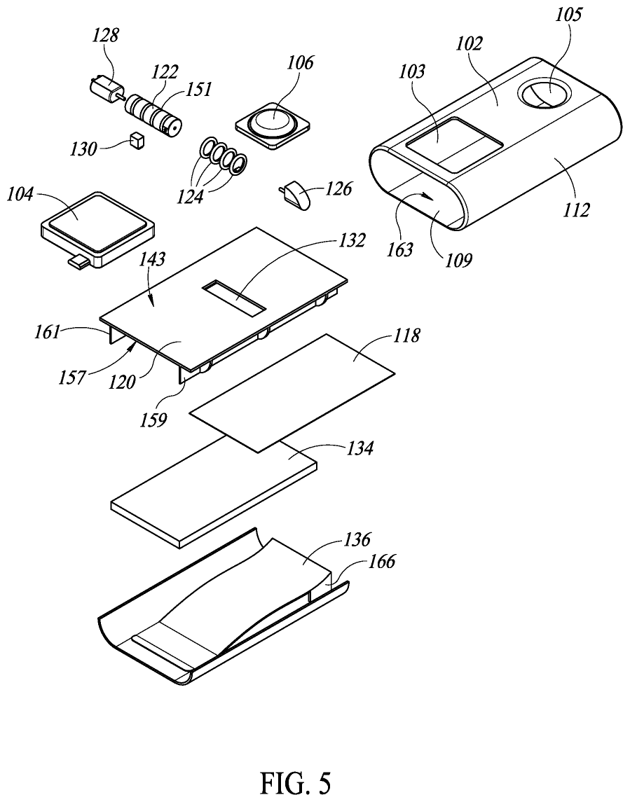

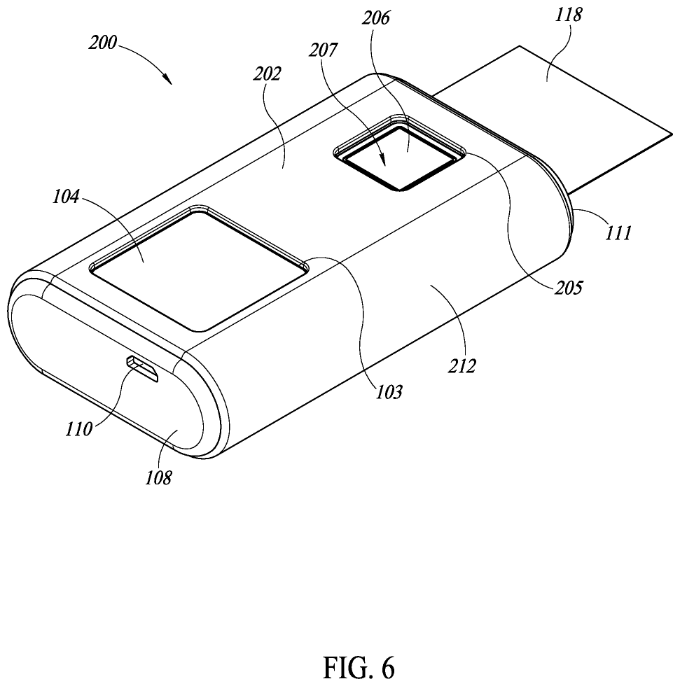

The first and second ends also include beveled or angled edges 133, 135. The angled edges 133, 135 extend between the outer shell 112 and a flat surface 137, 139. In FIG. 3A, which is a cross-sectional view along line 3A-3A, an opening 141 in the second end 111 is illustrated with the pharmaceutical agent 118 in an ejection action. The inner shelf 120 is centrally positioned within the device 100 and provides a support for the various devices and a guide for each strip or package of the pharmaceutical product as it is moved out of the device.

The display 104 may be mounted on a first surface 143 the inner shelf 120. The first surface faces the first flat surface 113 of the device. The display 104 is housed in a package that includes a variety of electrical components with external connections to couple to the other circuits of the device. In this embodiment, the processor 145 is housed in a same package as the display. A battery may be included in the same package as well. The package is coupled to the biometric sensor to provide power and to exchange data.

The device 100 includes a motor 128 and a rotation sensor 130 that are positioned within the outer shell. The motor 128 is coupled to and turns a roller 122 and the rotation sensor 130 detects the rotational position of the roller 122. The rotation sensor 130 then communicates the rotational position of the roller 122 to the processor 145 which controls the rotation of the roller 122. In some embodiments the roller 122 is supported axially by roller support 126 mounted on the inner shelf 120. In some embodiments the roller has a plurality of radial engagement loops 124 that are in contact with the pharmaceutical agent 118 through a shelf opening 132. In alternative embodiments the rotation sensor 130 detects the position of a shaft or a camshaft (not depicted) which has a plurality of radial engagement loops 124 that are in contact with the pharmaceutical agent 118 through a shelf opening 132.

The radial engagement loops 124 are O-rings or other suitable rubber or plastic members that are positioned within grooves 151 of the roller 122. The radial engagement loops 124 are positioned to extend into the opening 132 and contact a next pharmaceutical agent to be delivered at the authorized time interval or frequency to an authorized user.

When the inner shelf 120 is disposed within the outer shell 112 it forms a first compartment 153 that houses the display 104, the motor 128, the roller 122, the roller support 126, and the biometric sensor 106. In addition, when the inner shelf 120 is disposed within the outer shell 112 it forms a second compartment 155, which contains the pharmaceutical agent 118 and in some embodiments a cartridge 134, as depicted in FIG. 5, containing a plurality of doses of a pharmaceutical agent. The inner shelf 120 includes a second surface 157 with a first and second support 159, 161 extending transversely from the second surface. The first and second support 159, 161 interact with an interior surface 163 that is between the first and second curved surfaces.

The pharmaceutical agents 118 are positioned between the first and second supports. The cartridge 134 is also positioned between the first and second supports. A top-most pharmaceutical agent is positioned adjacent to the second surface of the inner shelf 120 so that the radial engagement loops are in contact with the pharmaceutical agent for ejection at the appropriate time.

In some embodiments, a spring 136 attached a mount 166 is configured to apply force to the pharmaceutical agent 118 to facilitate contact between the pharmaceutical agent 118 and the plurality of radial engagement loops 124 through the shelf opening 132 in the inner shelf 120. The spring 136 is coupled to the interior surface 163 and applies pressure to either the cartridge or the pharmaceutical agent.

In some embodiments the method of using the delivery device 100 includes the user interacting the biometric sensor 106 to turn on the display 104 and authenticate the user as the patient authorized to use the delivery device. The display 104 contains a processor and memory unit running software to authenticate the user. The authorized user then interfaces with the display to initiate the dispensing of an appropriate dose of the pharmaceutical agent 118. The processor operably linked to the motor 128 initiates the motor 128 to turn the roller 122. As the roller 122 turns the plurality of radial engagement loops 124 in contact with the pharmaceutical agent 118 through the shelf opening 132, the pharmaceutical agent is dispensed from the pharmaceutical agent delivery opening positioned at the first end of the delivery device 100. The motor 128 will continue the rotation of the roller 122 which is engaged with the pharmaceutical agent 118 by the radial engagement loops 124. The rotational displacement of the roller is monitored by the rotation sensor 130 and the data sent to the processor. The motor 128 will continue to rotate the roller 122 for a predetermined rotational distance configured to dispense a single dose of a pharmaceutical agent 118. This predetermined or threshold rotational distance may be such that the radial engagement loops 124 maintain contact with the pharmaceutical agent 118 being dispensed. This maintained contact is to avoid the pharmaceutical agent 118 from being completely and loosely dispensed where the user could mishandle and drop. The pharmaceutical agent 118 may be dispensed such that the user can grasp and pull it the remaining distance out of the delivery device 100. The user's pull force will easily overcome the contact resistance that the radial engagement loops 124 have on the pharmaceutical agent 118. The spring 136 coupled to the mount 166 applies force to the pharmaceutical agent 118 or alternatively the cartridge 134 to facilitate engagement with the radial engagement loops 124 through the shelf opening 132.

In one embodiment of the method of using a delivery device, such as device 100, a user will receive a prescription for a particular pharmaceutical agent 118 from an authorized health care provider. The health care provider or authorized pharmacy will load a delivery device 100 with the appropriate prescription. The health care provider will then associate the delivery device 100 with the patient, including the patient's demographic identifiers as well as at least one biometric identifier. In some embodiments this will be a fingerprint or a retinal scan. The health care provider will input the data into the delivery device through port 110 to be stored in the memory of the device. The health care provider will then give the delivery device 100 to the user to verify it is correctly associated with the patient.

When the patient is ready to receive their dose, the patient will input biometric identification into biometric sensor 106 to be identified as an authorized patient. The processor will process the biometric information to confirm the user is the authorized patient, then the processor will determine if the lockout period has elapsed and the pharmaceutical agent is available to be dispensed. The delivery device display 104 will turn on and will provide information regarding the dose and frequency to the patient along with demographic information. If the lockout period has not elapsed the display 104 will indicate the time until the dose is available, for example, in minutes. If the lockout period has elapsed and the pharmaceutical agent is available to be dispensed according to the prescribed dose frequency, the authenticated patient can initiate dispensing of the appropriate dose of the pharmaceutical agent 118.

In some embodiments the display 106 will also provide a count of the remaining doses in the delivery device 100. In some embodiments the display 106 will indicate a warning when the delivery device 100 is within in a specified time, such as a day or two, of running out of pharmaceutical agent doses. In some embodiments the patient will take a single dose per day and will require at least one visit to the health care provider per month to get the delivery device reloaded. When the patient returns with the device to the health care provider the health care provider will verify that the device is from the authorized patient and will download the usage data from port 110. The health care provider will verify that the device has not been tampered with and will reload a new cartridge of doses of a pharmaceutical agent for the patient. The health care provider may reload the device by unlocking the device and then securely locking it. In some embodiments the health care provider will take back the used device to be recycled for later use and will provide a new device to the patient. In this embodiment the patient's data will be scrubbed from the old device. In some embodiments the patient will only need to exchange the device by mailing it to the specialty online pharmacy or other authorized pharmacy which may improve access to the prescribed pharmaceutical such as in rural areas of the country where access to treatment for OUD is limited or nonexistent. Local authorized pharmacies may also have the capability to refill or exchange devices.

FIG. 6 is an alternative embodiment of a delivery device 200. The delivery device 200 has an outer shell 212 with a first end 108 opposite a second end 111 similar to the device 100 as depicted in FIGS. 1-2D. The shell 212 is has a plurality of openings, including a first opening 205 and a second opening 103 through the first side 202. The first opening 205 is aligned with a biometric sensor 206 when the delivery device 200 is in a closed or sealed configuration. The first opening 206 is rectangular as depicted in FIG. 6 and surrounds the biometric sensor 206. In some embodiments the biometric sensor 206 may be a fingerprint sensor that is supported on a substrate that is internal to the device 200. A portion of the sensor 206 will be housed within the outer shell, with an interfacing portion extending through the first opening 205. The biometric sensor 206 may be a fingerprint sensor having a square surface 207 positioned within the square shaped first opening 205.

In one embodiment, the finger print sensor may include a scanner having a light-sensitive microchip (either a charge-coupled device or a complimentary metal oxide semiconductor image sensor) to produce a digital image. Alternative embodiments, may use time of flight or other distance sensors sensitive enough to detect ridges on a finger. A processor or ASIC in the finger print sensor analyzes the image and implements pattern-matching to compare the user's finger print to an authorized user finger print stored in the device's memory.

In one embodiment, the patient provides the biometric information at a doctor's office, a clinic for addiction treatment, a pharmacy, or other biometric information collection location. This biometric information is collected and stored in a database that is accessible by an entity managing the distribution of the pharmaceutical devices and the kiosks. This database is configured to communicate the biometric data to each individual device through a wired communication mechanism or remotely through wireless transmission means, such as WiFi, Bluetooth, or cellular networks. The device will include a plurality of communication devices, such as through the data port in a wired configuration, or a wireless transceiver housed within the outer shell 212.

As noted above, embodiments of the present disclosure may have a plurality of biometric identification options that may be used in combination by the device and kiosk to ensure or minimize tampering or unauthorized acquisition of the pharmaceutical agents. In another embodiment, the device 200, may include a second sensor position between the display 104 and the biometric sensor 206 to add a second biometric identification option, such as a retinal scan or a keypad for entering a pre-programed code. The devices of the present disclosure are configured to be resistant to easy tampering to minimize unauthorized access to the pharmaceutical agents.

Returning to the outer shell 212, the first opening 205 of FIG. 6 includes a beveled or angled edge 207 that surrounds the biometric sensor and provide a transitioned edge for a user's finger. Corners of the square first opening 205 are rounded in this embodiment. In other embodiments, the corners may be substantially right or acute angles. The first opening 205 is closer to the second end 111 than the second opening 103. The second opening 103, similar to the second opening 103 from FIG. 1, is aligned with a display 104 when the delivery device 200 is in a closed or sealed, ready for use configuration.

In some embodiments, the delivery device 300 is configured to be worn on a user's wrist, as depicted in FIGS. 7A through 7C. The delivery device 300 has a wristband 313 coupled to the outer shell or package 312. The outer shell 312 has a first end 308 opposite a second end 311, where the outer shell 312 is disposed between the first end 308 and the second end 311. The outer shell 312 has a first side 302 opposite a second side 314, as depicted in FIG. 7C. In alternative embodiments, the first side 302 may be coupled to the second side 314 by a hinge such that the device opens like a clam shell instead of having the first and second ends. Secure locking features would be incorporated to minimize tampering. In some embodiments, a chemical or other self-destruction system may be included to make the pharmaceutical agent inert in the event of tampering.

In some embodiments the outer shell 312 has a plurality of openings, including a first opening 305 and a second opening 303 on the first side 302. Similar to the configuration seen in FIG. 1, the first opening 305 is aligned with a biometric sensor 306 when the delivery device 300 is in a closed or sealed configuration. The delivery device 300 has a display 304 aligned with the second opening 303. The biometric sensor 306 is used to authenticate the specific user associated with the device and to only dispense the pharmaceutical agent 118 when the specific user has been confirmed by a confirmation of identification process.

In some embodiments, the second side 314 has a plurality of biosensors to provide information on the authenticated user's vital signs that may include but not limited to heart rate and blood oxygenation. In some embodiments, the second side 314 is configured to provide haptic feedback to the authenticated user to indicate the device 300 is ready to dispense a pharmaceutical agent 118 at a specific prescribed frequency and dosage. In some embodiments the pharmaceutical agent is in the form of a film or strip. In some embodiments this film or strip can be 2.2 cm×1.3 cm, a variety of sizes depending on manufacturer, as unpackaged films or strips.

In some embodiments the device 300 can be worn on a user's wrist but will not dispense a pharmaceutical or provide patient information on the display 304 until the user is authenticated with the biometric sensor 306. The display 304 may be a touch screen or other electronic display to provide information and interact with the patient or user. For example, the display 304 may provide dosing, frequency, and biographical information of an authorized patient when the delivery device 300 is associated with the authorized patient. The display may provide information about battery life remaining and provide time and date information, including availability of the next dose according to the proscribed frequency.

The outer shell 312 includes a first flat surface 313 and a second flat surface 315, as depicted in FIG. 7C. The first and second flat surfaces 313, 315 have a first dimension 317 in a first direction D1. The first and second flat surfaces 313, 315 are spaced from each other by a second dimension 319 in a second direction D2. The outer shell 312 includes a first curved surface 321 and a second curved surface 323. The curved surfaces are coupled to and integrally formed with the first and second flat surfaces 313, 315 in one embodiment. A third dimension 325 in the first direction, between an outermost point of the first and second curved surfaces is greater than the first dimension 317.

The outer shell includes a fourth dimension 327 between the first end 308 and the second end 311. The fourth dimension 327 extends in a third direction that is transverse to the first and second directions. The fourth dimension 327 is greater than the third dimension 325. A radius of the first opening 305 from the outermost edge of the bevel is a fifth dimension 329. The display in the second opening 303 includes a sixth dimension 331 that is greater than the fifth dimension 329. The fifth and sixth dimensions extend in the third direction.

The first and second ends are physical components that couple to an interior surface of the outer shell 312 to securely and tightly seal the delivery device 300. The second end 311 of the delivery device 300 is configured to deliver a pharmaceutical agent 118. In some embodiments the pharmaceutical agent 118 is an individually wrapped or unwrapped medication, such as SUBOXONE®. The first end 308 of the delivery device 300 includes a data and charging port 110 that is operably linked to a processor or application specific integrated circuit and a battery. In some embodiments, the data port 110 may be a USB-C port. The data port 110 enables an authorized user to download or upload data to and from the delivery device 300 using encryption techniques. In some embodiments, the delivery device 300 is charged through the second side 314. In some embodiments, data is transferred to the delivery device by wireless data transfer, such as through BLUETOOTH®.

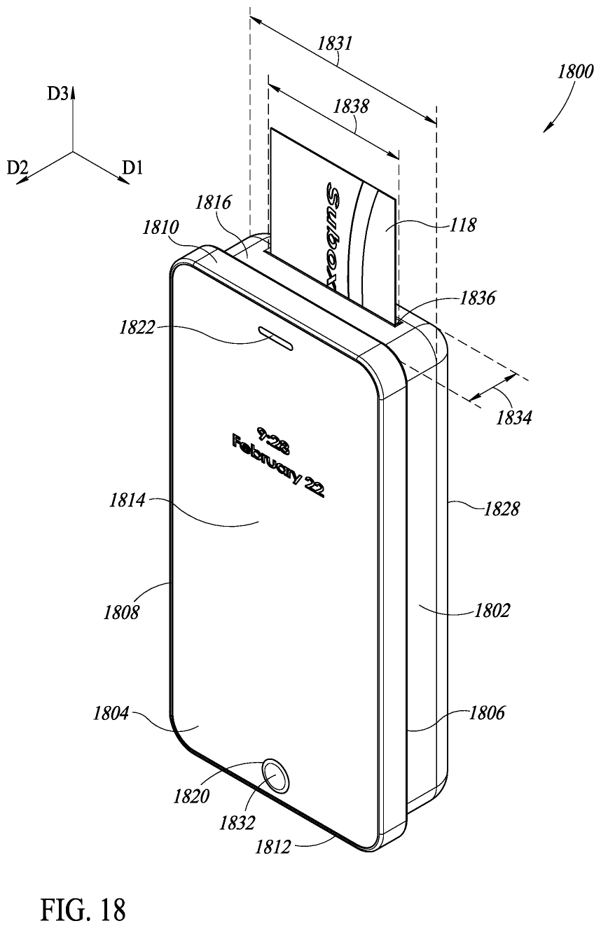

The confirmation of identification process that can be utilized with any of the devices of the present disclosure is described below. The confirmation of identification begins as the patient starts managing their own treatment. The treating physician or healthcare provider will establish a treatment plan, including frequency and dose for the patient's access to the pharmaceutical agent. The treatment plan is either written on a prescription and shared with a pharmacy or entered into a database, or electronically submitted, such as medical information system 1706 in FIG. 17.

The healthcare provider selects frequency and dose based on the patient's level of addiction, their body size and composition, and progress through their treatment plan, among other things. As an example, some patients may receive a dosing frequency of a strip every twelve hours. In some instances the dosing window may be more frequent than twelve hours. In some instances the dosing window may be less frequent than twelve hours. It is noted that different states and counties have different rules and regulations regarding acceptable availability, as such, the devices can be programmed to honor local rules and regulations accordingly.

The patient provides their biometric identification information to either the treating physician, the healthcare provider, the pharmacy, or other authorized institution (i.e. healthcare provider locations 1702), including at the kiosk, or on a mobile application, to create their patient profile to be stored in the medical information system 1706. The patient may also create a pin that is one of the security checks performed during a patient authentication sequence on the device or the kiosk or both.

The patient is provided a first device either from the location where the biometric identification is gathered, from a kiosk, an OTP, authorized provider, a local authorized pharmacy, or it may be mailed by a specialty online pharmacy. The biometric identification and the patient treatment plan, including dosing and frequency may be stored in the patient profile and transmitted either wired or wirelessly to the distribution kiosks 1704, the medical information system 1706, and the healthcare provider locations 1702 through a network 1708, see FIG. 17 for more details.

As the patient is away from the healthcare provider locations and on their own during their treatment, the device will be tracking time between doses to accurately provide a dose to the patient at an appropriate time interval. If the patient activates the device before the end of the time period between doses, the device will display a message that no pharmaceutical agent is available at this time. The message may also include a statement of the amount of time remaining (i.e., the lockout period) before the next dose is available. The device may be configured to track the number of attempts of the patient to access a dose during the lockout period to be reviewed by the healthcare providers at a follow up or check in. This data can be transmitted wirelessly, such as once a day to the medical information system 1706 or when connected to a WiFi network or a cellular network. Alternatively, if the patient does not have access to wireless networks, this data may be transmitted when the device is returned to a kiosk or plugged into a kiosk for charging.

When it is within an acceptable threshold time period of the next dose, such as 10 minutes, the device may make a sound or other alert, like vibration to notify the patient that a dose is available. These devices may include a piezoelectric chip or other vibration producing chip within the outer shell to provide an alert.

The device will not release a dose until the authentication sequence has been validly completed. The authentication sequence will include one or more biometric or user identification checks, such as a password and a valid fingerprint or retinal scan of the patient. The authentication sequence could also include a voice recognition option, using the microphone. The patient may have previously recorded a phrase that is stored in their patient profile to be compared when accessing the device.

When the timing is correct and the biometric identification has been verified by comparing the inputs from the patient to the patient profile, the dose will be released. In some devices, there may be additional sensors on the device to detect oxygenation, heart rate, and other vitals that can be used at the time of access or throughout the lockout period to track the patient's medical status. This information may be analyzed by the healthcare provider when determining adjustments to dosage and frequency as cravings and their frequency may be relevant to the treatment plan. If a device is connected wirelessly, the device can send such information in real time, which could be processed in the medical information system 1706 and alert the healthcare provider or emergency medical services when the patient's status is outside of a safe parameter.

Once the patient has used all allotted doses in the first device or a subsequent device or if the device is malfunctioning, the patient can return to the healthcare provider for the second or subsequent device, a kiosk (described in more detail below), a specialty online pharmacy, or a local authorized pharmacy. When the last dose is delivered, the display may indicate that no further doses are available to let the patient know to take action for a replacement. As the device gets close to having no doses remaining, the device can provide a message to the patient that a limited number of doses are remaining. This may help remote patient be prepared to find transportation to the closest kiosk or healthcare provider a local authorized pharmacy, or coordinate exchange through an online specialty pharmacy. In some embodiments, the device can provide location information of the closest kiosk through the network 1708.

In another example, the devices can share when the patient is not receiving the doses at the prescribed frequency. In the wearable configuration that includes sensors for vitals tracking, the device, when in wireless communication enabled environments, can transmit the vital information in real-time to the healthcare provider through the network. The medical information system may include threshold vital measurements that are indicative of an overdose or other medical emergency. If such threshold vital measurements are exceeded, the device may be instructed to alert and communicate with the patient such as through a message on the display. If the patient does not respond within a specified time period or does not select a confirmation of safety icon, the device may call or alert a local emergency department or 911.

Both the hand-held device and the watch or wearable device may configured to include global positioning systems or other location based systems to identify a present location of the device and identify the nearest kiosk for a refill or telemedicine interaction. In additional embodiments, the wearable and hand-held devices are configured to connect via the network to remote health care providers or an entity that manages a mail-order refill/exchange system. The biometric information will be universally recognized by all kiosks and devices, meaning that if a patient is traveling and accesses a kiosk in a different location, the information will be recognized and interoperable.

FIG. 17 is a block diagram of a medical treatment system 1700, according to one embodiment. The medical treatment system 1700 includes healthcare provider locations 1702, distribution kiosks 1704, and an information system 1706. The healthcare provider location 1702, the distribution kiosks 1704, and information system 1706 are communicatively coupled together by one or more networks 1708. The components of the medical treatment system 1700 cooperate together to provide medical services to individuals. The distribution kiosks 1704 may be any one of the kiosks or combinations of the kiosks described in more detail below.

In one embodiment, the healthcare provider locations 1702 can include hospitals, medical clinics, pharmacies, urgent care centers, emergency rooms, or other locations or venues at which healthcare professionals may provide healthcare services to individuals. The healthcare provider locations 1702 include computing resources. The computing systems can include processing resources, memory resources, data transmission resources, displays, input devices, or other systems and devices that may be utilized by healthcare professionals.

In one embodiment, the computing systems can store medical information related to patients that utilize the services of the healthcare professionals. The medical information can include personal identification information such as names of patients, addresses of patients, insurance information of patients, birthdates of patients, government identification numbers of patients, or other types of personal identification information associated with patients. The medical information can also include medical history information. The medical history information can include data related to previous medical conditions of patients. The medical history information can include information related to previous or ongoing ailments, injuries, sicknesses, diagnoses, treatments, surgeries, allergies and medications. This information may be stored in one or more databases in computer memories at least partially located at the healthcare provider location. The medical information may include the patient identification profile, including finger prints, retinal scans, or any other biometric identifier suitable for these applications.

In one embodiment, an individual may consult a healthcare professional, such as a physician. The physician may make a diagnosis of a condition of the individual, such as opioid use disorder (OUD). The physician may prescribe a medication to the user to be administered to the user via a pharmaceutical agent delivery device, such as strips of the pharmaceutical agents described above, among other treatments. The physician may provide the pharmaceutical agent delivery device (such as the devices 100, 200, etc. described above) to the user. Alternatively, the physician may indicate that the pharmaceutical agent delivery device is to be received by the user from a healthcare professional of a separate healthcare provider location. In one example, the second healthcare provider location is an online specialty pharmacy or addiction treatment facility.

In one embodiment, the physician, or another healthcare professional associated with the physician, may record details of the diagnosis and prescription in the computing systems of the healthcare provider locations. The details can include a name of the medication, dosage levels to be administered, a schedule for adjusting dosage levels, a number of allowable refills of the medication, contingencies for replacing the medication with a second medication, dosage levels of the second medication, and other details related to the medical condition of the individual, the diagnosis, and the prescription. If the physician provides the pharmaceutical agent delivery device to the user, then the physician or other healthcare professional may record, in the computing systems, an identification of the pharmaceutical agent delivery device.

In one embodiment, after receiving the diagnosis from the physician, the individual may go to a pharmacy to obtain the pharmaceutical agent delivery device. The pharmacist, or another healthcare professional, may provide the pharmaceutical agent delivery device to the user. The pharmacist or other healthcare professional may record, with computing systems associated with the pharmacy, details related to the individual, the diagnosis of the individual, the prescription, medical history of the individual, details related to the pharmaceutical agent delivery device, or other suitable information. This information may be stored at least partially in databases implemented, at least partially, in computer memories located at the pharmacy.

In one embodiment, some of the information associated with the individual may be stored with the medical information system 1706. The medical information system 1706 can include computing resources including, but not limited to servers, processing resources, memory resources, and data transmission resources. The medical information system 1706 can include physical and virtual computing resources.

In one embodiment, the medical information system 1706 can be, at least partially, a cloud-based medical information system. The medical information system 1706 can include medical information databases, medical information applications, and other medical information resources implemented with the computing resources. These databases, applications, and resources may be implemented partially or entirely in the cloud. These databases, applications, and resources may be accessible via the cloud.

In one embodiment, the previously mentioned information stored in association with the healthcare provider location 1702 may be partially or entirely stored in the medical information system 1706. In other words, personal identification information, medical history information, diagnosis information, treatment information, prescription information, or other information associated with individuals that is recorded at the healthcare provider location 1702 may be stored in the medical information system 1706. Some or all of the information may be duplicated at the healthcare provider location 1702 and the medical information system 1706. Alternatively, some or all of the information recorded at the healthcare provider locations may only be stored with the medical information system 1706.

In one embodiment, the medical information system 1706 and the computing systems of the healthcare provider locations 1702 may share software applications. The shared software applications may enable data stored at a healthcare provider location 1702 to be automatically sent to them recorded in the medical information system 1706. Likewise, the shared software applications may enable healthcare providers at the healthcare provider location 1702 to retrieve medical information related to individuals or devices from the medical information system 1706.

In one embodiment, an individual that receives a pharmaceutical agent delivery device will visit one of the distribution kiosks 1704 in order exchange one pharmaceutical agent delivery device for another. For example, if the medication in the individuals pharmaceutical agent delivery device has been entirely used, the user may access one of the distribution kiosks 1704 to exchange the empty pharmaceutical agent delivery device for a full pharmaceutical agent delivery device. Alternatively, the individual may exchange the pharmaceutical agent delivery device including one type medication, for another pharmaceutical agent delivery device containing a different type of medication.

In one embodiment, the distribution kiosks include computing resources such as processing resources, memory resources, data transmission resources, or other types of computing resources. These computing resources can receive, process, and store data related to pharmaceutical agent delivery device that have been provided to individuals. The computing resources of the distribution kiosks 1704 can include personal identification information, medical history information, and current diagnosis and prescription information associated with individuals. The computing resources of the distribution kiosks 1704 may include databases that store these types of information. Various encryption and data security methods will be implemented to ensure safe storage of any personal information. In some embodiments, the personal information will not be permanently stored in the kiosk and instead will only be accessed and temporarily saved at the kiosk when needed to interact with that patient.

In one embodiment, the computing resources of the distribution kiosks 1704 can assist in implementing security measures related to the distribution kiosks. For example, the computing resources of the distribution kiosks 1704 can assist in authenticating individuals and referencing databases to ensure that individuals are authorized to exchange or receive pharmaceutical agent delivery device. The computing resources can assist in ensuring that the identification of a pharmaceutical agent delivery device that a user seeks to exchange for another corresponds to the device that was legitimately provided to the user. If the identity of the pharmaceutical agent delivery device does not match the identity of the pharmaceutical agent delivery device provided to the user, then the distribution kiosk 1704 may refuse to exchange the pharmaceutical agent delivery device for another.

In one embodiment, the computing resources of the distribution kiosks 1704 may assist in obtaining and analyzing current physical indicators of the user. These indicators may include heart rate, blood pressure, pupil dilation characteristics, body temperature, facial features, or other indicators of the current physical state of the user. The distribution kiosks 1704 may include sensors to sense these physical indicators. The computing resources of the distribution kiosks 1704 may then assist in performing diagnostics based on the sensed physical indicators.

In one embodiment, the computing resources of the distribution kiosks 1704 may include one or more machine learning-based analysis models. The machine learning-based analysis models may be trained, with one or more machine learning processes, to determine whether a user should receive the pharmaceutical agent delivery device having the particular medication. The machine learning processes may train the analysis model to make these determinations based on the current physical indicators of the user.

In one example, when a user accesses a distribution kiosk 1704 to attempt to exchange one pharmaceutical agent delivery device for another, sensors of the distribution kiosk 1704 may sense current physical indicators of the user. These current physical indicators may pass to the analysis model. The analysis model may determine that based on the current physical state of the user, the user should not receive a pharmaceutical agent delivery device having the first medication. Instead, the analysis model may determine that the user should receive a pharmaceutical agent delivery device having a second medication, or that the user should not receive a pharmaceutical agent delivery device at all.

In one embodiment, the distribution kiosks 1704 stored data related to the diagnosis and prescription of individuals. When a user receives a pharmaceutical agent delivery device from the distribution kiosks 1704, the distribution kiosk 1704 encodes medication distribution parameters based on the diagnosis and prescription information. This can include dosage rates, dosage timings, or other data related to the distribution of the medication.

In one embodiment, the distribution kiosk 1704 stores the same types of data as previously described related to the healthcare provider location 1704 and the medical information 1706. The databases of the distribution kiosks 1704 may store all of this data, part of this data, or other types of data not stored at all in the healthcare provider locations 1704 and the medical information system 1706. The computing resources of the distribution kiosks 1704 may implement software applications that communicate with the healthcare provider location 1702 and the medical information system 1706 in order to retrieve information from the healthcare provider locations 1702 and the medical information system 1706.

In one embodiment, the networks 1708 enable communication between the computing systems of the healthcare provider locations 1702, the computing resources of the distribution kiosks 1704, and the medical information system 1706. The networks 1708 can include the combination of one or more of local area networks, wireless area networks, cellular networks, satellite communication networks, the Internet, or other types of communication networks.

In one embodiment, healthcare provider location 1702 retrieve, when needed, medical data from the medical information system 1706 related to patients rather than storing that data permanently. The healthcare provider location 1702 can also provide data to the medical information system 1706 and to the distribution kiosk 1704. The networks 1708 enable this retrieval and transmission of data.

In one embodiment, when a user attempts to access a distribution kiosk 1704, the distribution kiosk 1704 retrieves data related to the user and/or the users pharmaceutical agent delivery device from one or both of the healthcare provider location 1702 and the medical information system 1706. The distribution kiosks 1704 may also provide data related to a transaction with a user to one or both of the healthcare provider location 1702 and the medical information system 1706. The distribution kiosks 1704 transmit and receive this information via the networks 1708.

### Kiosk System

The kiosk system will have the ability to remotely perform the complex functions conducted by an opioid treatment program (OTP) clinic. These functions include conducting telemedicine consultations, receiving electronic prescriptions in real time from health care providers, dispensing prescribed pharmaceutical agents, such as buprenorphine or methadone, live videos feed during observed daily dosing, ability to verify a patient's identity, confirming that the daily dose has been ingested and not diverted, and dispensing take-home doses to authorized users.

In some embodiments, a kiosk system may require a health care provider through a telemedicine consultation to authorize the dispensing of a prescribed pharmaceutical agent or take-home doses to authorized users. In some instances, take-home doses may be dispensed directly from the kiosk or from a secure take-home locker system associated with the kiosk system. This secure take-home locker system may be attached to the kiosk or a complete separate assembly operably linked to the kiosk system.

In some embodiments, a liquid pharmaceutical agent may be dispensed into an appropriate receptacle with a pump. A pump may dispense according to the patient's specific electronic prescription located in the medical information system. A pharmaceutical agent or receptacle may be labeled with patient specific information by means of a printer or other functional means before being dispensed. For example, take-home doses may be dispensed into bottles, sealed child-proof, then labeled with prescription information for the specific patient before being fully dispensed. In some instances where a pharmaceutical pump is used, a flushing and cleaning routine may be initiated at certain intervals to maintain system cleanliness and pump calibration.

A specialty kiosk system 400 in FIG. 8, is designed specifically to exchange used and new pharmaceutical agent delivery devices (such as those described with respect to FIGS. 1-7C). This kiosk system 400 may also provide immediate telemedicine to those that need treatment for opioid use disorder (OUD). The specialized kiosk may dispense pharmaceutical delivery devices that will dispense prescribed pharmaceutical agents in the form of films, strips, transmucosal products, or patches, at the prescribed dose and frequency, to the securely identified authorized patient. The specialized kiosk may also dispense single doses—including those for emergency cravings, or a short-term supply of the prescribed opioid for the treatment of OUD securely to an identified authorized patient. The kiosk system may also dispense naloxone (a prescription drug used to reverse the effects of opioids) as well as the long-acting injectable buprenorphine (e.g., SUBLOCADE®). Some specialized kiosk systems may provide telemedicine including dispensing medications or devices, and some kiosks may just dispense medications or devices to the securely identified user. The kiosk will have “smart inventory” which actively communicates that particular kiosks inventory count to the party responsible for refilling to eliminate the possibility of running out of specific devices, medications, etc.

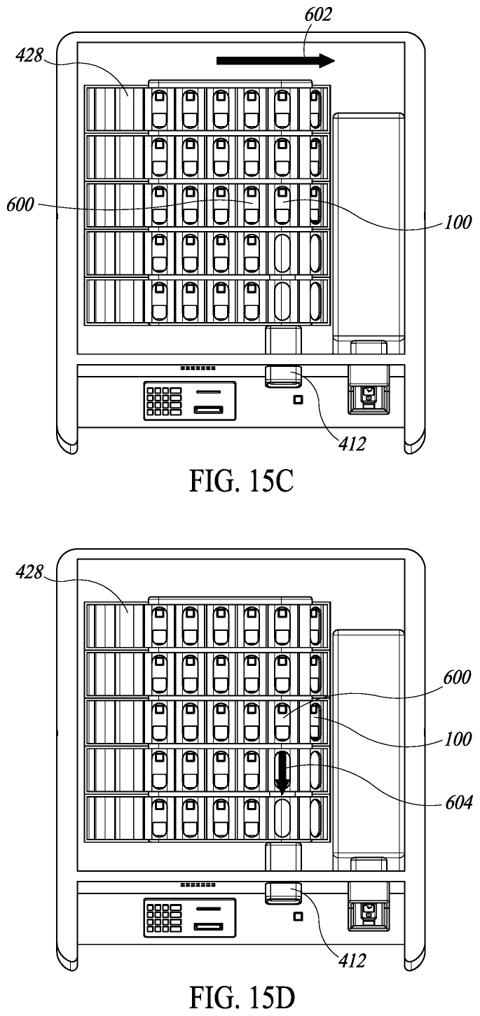

The kiosk system 400 in FIG. 8 includes a patient facing side 401 that includes an interface or data entry area 403. The patient facing side 401 includes an angled surface 402 that supports a plurality of user interfaces including, but not limited to a camera and retinal scanner 408, a display 406, a microphone and speaker 405, a pharmaceutical agent delivery device exchange port 412, and a charging or docking station 416, 418 (see FIG. 10).

In some embodiments the kiosk system 400 may have a dispensing mechanism 414, depicted in FIG. 10, configured to provide a single dose of a pharmaceutical agent, such as pharmaceutical agent 118, which may be buprenorphine, long-acting injectable buprenorphine (e.g. SUBLOCADE®, BRIXADI®, OR BRAEBURN®), methadone, or naloxone. In some embodiments the first docking station 416 is configured to interact with a wearable pharmaceutical delivery device, such as delivery device 300 depicted in FIG. 6. In some embodiments the second docking station 418 is configured to interact with a pharmaceutical delivery device, such as delivery device 100 depicted in FIG. 1. The second docking station 418 may interact with the data port 110 to enable the upload or download of data from the delivery device 100. These stations 416, 418 can be utilized for charging the devices if the patient does not have consistent access to stable housing, such that even if the device still has doses remaining, the patient may use the kiosks to charge their devices. In some instances the docking station 416 is configured to dispense a refill cartridge to be used with the device 100. The refill cartridge (not depicted) will also be resistant to tampering and water proof. The refill cartridge may also contain a tamper sensor that would render the pharmaceutical agent within the cartridge inert if the cartridge is tampered with.

When the devices are placed in to the stations 416, 418, any data collected by the device during the period since the device was last connected to the network 1708 may be transmitted through the kiosk to the medical information system 1706. Such data can include the number of attempts to acquire a dose between authorized dose time periods. The data can include vitals collected by the sensors on the watch-based or wearable device, etc. The kiosk's software will include automated decisioning (AD) software paired with the biometrics and other sensors, such as video cameras. The AD software will facilitate efficient and safe patient interactions by utilizing the biometric and other sensor data.

The kiosk and the user interfaces are configured to be water resistant and tamper resistant. In some embodiments the display 406 is an interactive touch screen. A second angled surface 425 that is transverse to the first angled surface 402 supports the keyboard 404 and a fingerprint or other biometric identification sensor 409. In some embodiments, the keyboard 404 also has a card reader 433 or scanner to identify an authorized user. For example, a pharmacy may issue a specific card when collecting the biometric identification information to be associated with a specific patient, such that a retinal scan or a finger print and the specific card are required before the device 100 is ejected from the kiosk. This is one possible set of factors to be evaluated before the device 100 is delivered to the user. The identification information may be stored in a chip in the card, an RFID device, in a magnetic strip, or other suitable mechanism for storing data.

The kiosk also includes a third angled surface 411 adjacent to a top side 413. This third angled surface 411 includes or supports a camera 410 at an orientation that has a field of view of the patient. This camera may be used for security purposes and may be used for facial recognition in the identification sequence of the authorized user.

The kiosk 400 includes a first and second side 415, 417 that are coupled to the top 413 and a bottom. Each of the first and second side 415, 417 include a privacy shield 407 configured to improve the security of the user interface from observation by a third party. The privacy shield 407, in one embodiment, includes a curved edge 419, 421 that extends from the top 413 to the bottom of the kiosk 400. The curved edge may include a convex portion closer to the top and a concave portion closer to the bottom.

The kiosk 400 includes a panel 423 that is coupled to the second angled surface 425, is transverse to the second angled surface 425, and is between convex parts of the curved edges of the first and second sides 419, 421.

The specialized kiosk may include encrypted secure identifiable information connecting the authorized patient to the device or medication dispensed. Alternatively, this information will be stored remotely in the cloud as discussed with respect to FIG. 17. The device will be configured to only dispense the correct pharmaceutical agent to the authorized patient after secure identification. Examples of identification strategies may include, but are not limited to, fingerprint, handprint, palm veins, DNA identification, face-recognition, retina identification, other biometric identification, multi-source identification software, a personal identification number, or any combination of the foregoing. In some embodiments the retinal identification sensor 408 is used, as depicted in FIG. 8. Once the biometric information is obtained, it is universally recognized by all kiosks, devices, and the software application.

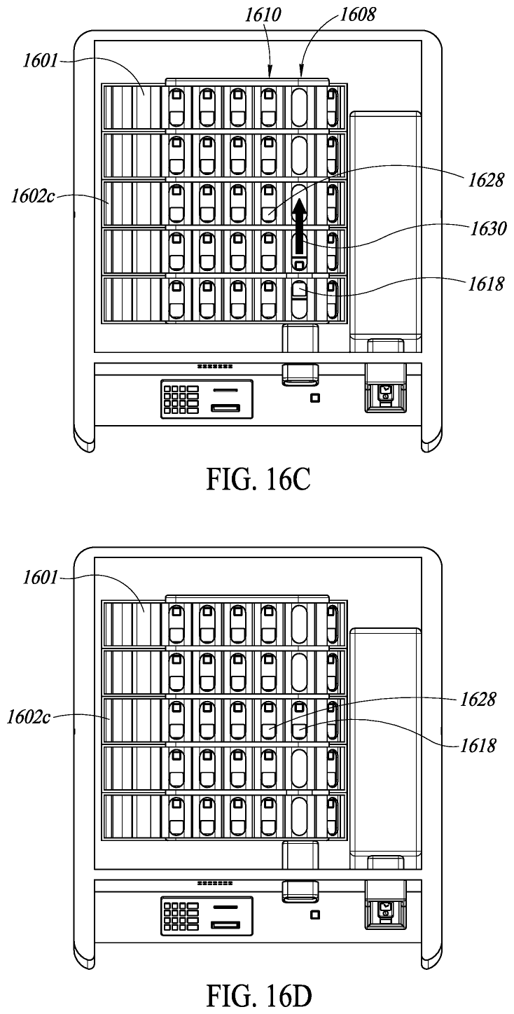

The kiosk system may include a dispensing mechanism 412 to load a device, see FIG. 10 and cross-sectional view in FIG. 14 through the line B-B of FIG. 10. The dispensing mechanism 412 extends from an exterior environment into the kiosk into an interior environment 435. The angled surface 402 is part of a wall 437 having a thickness 439. The dispensing mechanism or receptacle 412 includes a first exterior side 441 and a second interior side 443. A dimension 445 of the first exterior side 441 from the angled surface 402 to a patient interfacing end 449 is smaller than a dimension 447 of the second interior side from the wall 437 to an interface with a belt system 428. In this embodiment, the dispensing mechanism includes an oval opening sized and shaped to receive the device 100. Within the dispensing mechanism there is a security latch or wall that opens when an authorized user has requested to return one of the devices.

In some embodiments, the docking stations 416 and 418 are operably linked to a central processing unit (CPU) or mother board 422 that includes at least a processor, a memory, and wireless transmission and reception chips. This CPU will include a networking unit that enables the kiosk to connect both to the pharmaceutical delivery devices, the network 1708, and the internet or Wi-Fi to enable an authorized patient to download data from the pharmaceutical delivery device for a health care provider to access. This CPU 422 also enables a health care provider to upload data to the pharmaceutical delivery device. In some instances the CPU 422 will run an automated decisioning (AD) software, as described in more detail below.

This loading system or docking station in conjunction with the CPU 422 may include identification encryption methods to securely connect the contents of the devices and medical information to and associated with the authorized patient. In the loading system embodiment, where the docking station 416, 418 includes a refilling feature, the loading system may also provide an electronic, magnetic, or some other mechanism that may securely lock and unlock the device for unloading and reloading. The loading system may be implemented for both the wearable and hand-held device embodiments. When the patient places the wearable or device, in the loading system 416, 418, the kiosk will lock the device or wearable into place. The kiosk, after confirming this is an authorized user and identifying the dose and treatment plan will open the watch or device to remove the spent cartridge and insert a new cartridge. In some embodiments, this may be fully performed within the kiosk, out of sight of the patient. The device may be loaded with a prefilled cartridge, as previously described. This loading system may include identification encryption methods to simultaneously and securely connect the device, cartridge, and its contents to the authorized patient, including programming the frequency of doses and other treatment parameters to be displayed and tracked by the device during the next period of use. The devices dispensed may be disposable, or may be refillable and reusable.

The loading device or docking station in the kiosk system may be configured to receive a replaceable cartridge or the delivery device itself for loading of medications and identity encryption. Also, the loading device or docking station may be operable to communicate information associated with the prescribed opioid, device, or patient. In some embodiments the software in the kiosk system may communicate via wireless internet or via BLUETOOTH.

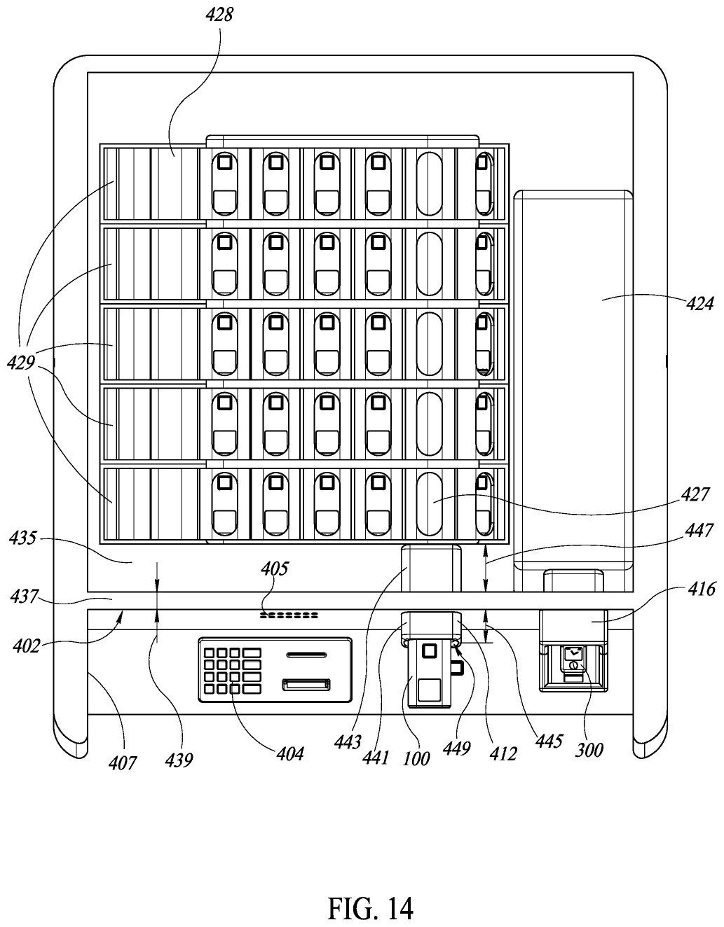

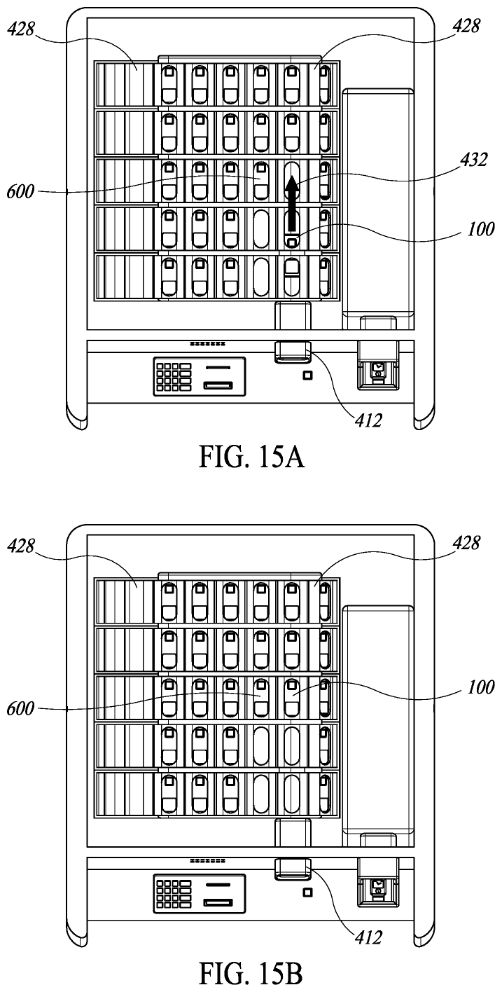

The kiosk may be highly secure, solidly built, and may also render the medication inert if tampering occurs with the kiosk. The kiosk may also include drug testing analyzers such as, for example, but not limited to, capillary blood sensors, biosensors, and drug breathalyzers to test for opioid levels and other controlled substances. These kiosk biosensors may test urine, blood, saliva, hair, or any combination of the foregoing to conduct drug level testing. For example, the telemedicine aspects of the kiosks may be conducted in a secure soundproof booth as shown in FIGS. 9A and 9B with secure technology allowing telemedicine to be performed as well as methods for obtaining biometric identification and some limited sample analysis. In some embodiments the entire kiosk 400 may be inside a privacy booth system 500 as depicted in FIGS. 9A and 9B. The privacy booth system 500 may have a security door 502 operably linked to a user interface 508. The user interface 508 may have a keyboard, biometric sensor, card reader, or any combination thereof to authenticate the user to unlock the security door 502. The interior 504 of the privacy booth system 500 may also have a seat 506 to allow the user to sit while using the kiosk system 400.

The specialized kiosk may be resistant to tampering, such as, but not limited to an ATM machine. The kiosk may be built to be weatherproof so that it can be housed outdoors. Some kiosks may provide dispensing only functions with the option of a drive-through. In some embodiments the kiosk system may be configured for both dispensing—pharmaceutical agents and dispensing devices—and telemedicine. The telemedicine booth may have soundproof doors that will facilitate private communication between an authorized patient and a health care provider. This may be a live health care provider or this could be an artificial intelligence system that includes chat bot messaging and a sequence of vital testing to determine a status of the patient in the booth. The telemedicine booth of the kiosk may include user interface devices such as a touch screen interactive computer, software to transmit biometric identification, internet connectivity, memory, video recording, phone, blood pressure cuff, pulse oximeter, technology to administer injectable medications, and a power source. The kiosk system may include a dispensing mechanism to provide a pharmaceutical agent, such as buprenorphine, as part of the telemedicine visit. The kiosk may interact with the patient using voice. The kiosk may also release a unique aerosolized scent within the kiosk booth and or dimmed lighting with blue back lighting to provide a therapeutic experience.

The kiosk 400 can be accessed by authorized personnel to replace or refill the inventory in the kiosk. The kiosk may be opened, for example, one the first and second side 415, 417 to access the internal components inside the kiosk. The authorized personnel may then perform any necessary maintenance or refill either the devices, retrieve empty devices, refill individual pharmaceutical doses, or replacement cartridges.

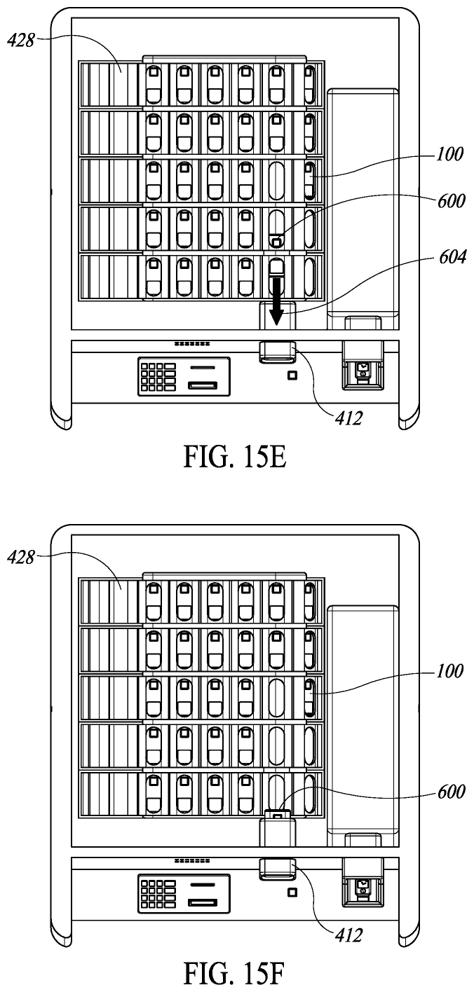

In FIGS. 10 and 11, the pharmaceutical agent delivery device exchange port 412 can be seen in conjunction with other elements of the kiosk 400. FIG. 11 is a cross-sectional through line A-A of FIG. 10. In some embodiments the kiosk system 400 includes the dispensing mechanism 414, configured to provide a single dose of a pharmaceutical agent, such as pharmaceutical agent 118, which may be buprenorphine, long-acting injectable buprenorphine (e.g. SUBLOCADE®), or naloxone. The dispensing mechanism 414 is operably linked or coupled to a single dose distribution subsystem 420. The distribution system 420 may also be coupled to the docking station 416 that may replace a used cartridge in a watch-based or wearable device. The distribution system 420 is configured to contain a plurality of pharmaceutical agents for single dose delivery, including injectable anti-overdose treatments. If the kiosk includes injections, a needle disposal compartment and port will be included in the patient interfacing surface for safe disposal of the used needle after use.

In some embodiments the first docking station 416 is configured to interact with a wearable pharmaceutical delivery device, such as delivery device 300 depicted in FIG. 6. In some embodiments the second docking station 418 is configured to interact with a pharmaceutical delivery device, such as delivery device 100 depicted in FIG. 1. The second docking station 418 may interact with the data port 110 to enable the upload or download of data from the delivery device 100. The first docking station 416 includes a charging or data exchanging prong 455. The prong is configured to support the device in a position to remove a used cartridge and receive a new cartridge through an interface and opening 457 through wall 437. The first docking station 416 includes an exterior portion 459 that has a base 461 that supports parts of the watch for charging, data exchange, cartridge exchange and other suitable purposes.

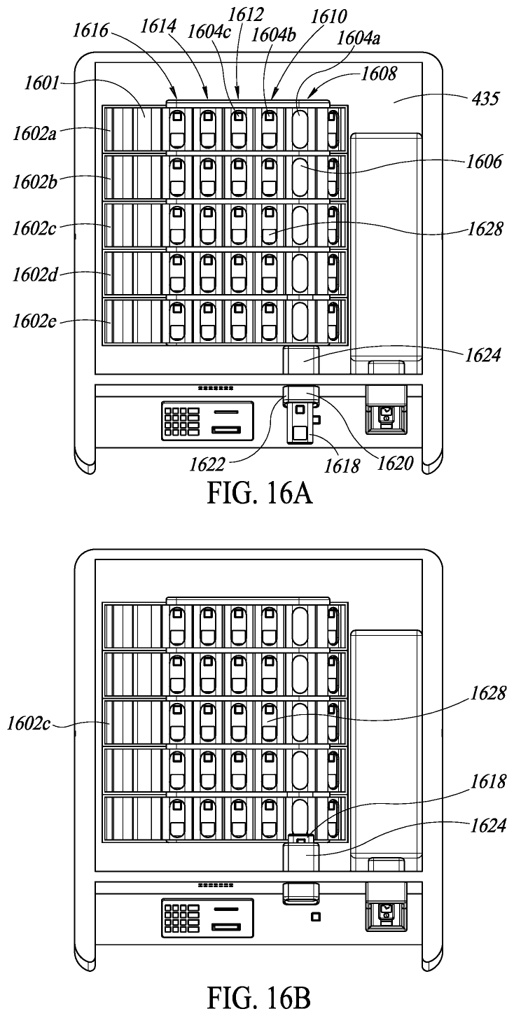

The distribution system 420 is configured to hold a plurality of pharmaceutical agents for single dose delivery through the dispensing mechanism 414. The CPU 422 is operably linked or coupled to the docking stations 416 and 418. In FIG. 13, the dispensing system 420 is illustrated as two separate systems, a first system 420a coupled to the first docking stating and a second system 420b coupled to the single dose dispensing mechanism 414. These systems may be implemented separately or together.

In some embodiments the display 406, cameras 410 and retinal scanner 408, the first docking station 416 and second docking station 418, as well as the device exchange port 412 and dispensing mechanism 414 are all coupled to CPU 422, which runs software that enables the kiosk system 400 to authenticate the user and provide the correct pharmaceutical agents or medical responses and communicate with the network 1708 in the cloud. In some embodiments the CPU 422 may include a processor and memory unit that are separate. In some embodiments the kiosk system 400 contains a networking unit (not depicted) to connect the kiosk system to the internet, cellular networks, or the network 1708. The CPU 422 may be housed in one place or separate motherboards may exist within the kiosk to manage and control the different systems within the kiosk.

In some embodiments the docking stations 416 and 418 are operably linked to the CPU 422 which may alternatively contain a networking unit that enables the kiosk to connect both to the pharmaceutical delivery devices and the internet or Wi-Fi to enable an authorized patient to download data from the pharmaceutical delivery device for a health care provider to access. CPU 422 also enables a health care provider to upload data to the pharmaceutical delivery device.

In FIG. 11, the second docking station 418 for the hand-held device includes a base 463 onto which a device 100 rests when engaged with electronic components in the second docking station 418. An opening 465 through the wall 437 provides an interaction area for the device to engage with the CPU 422 and cartridge exchange component 424.

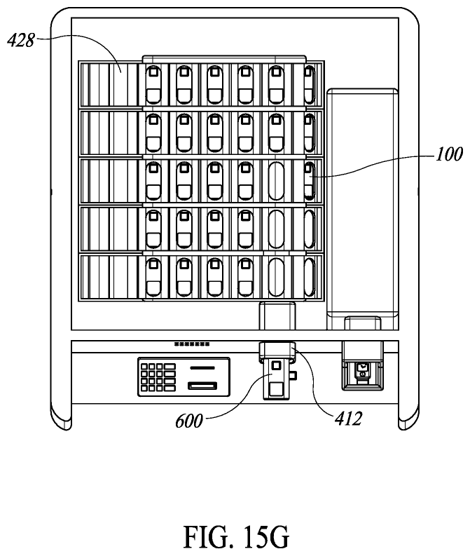

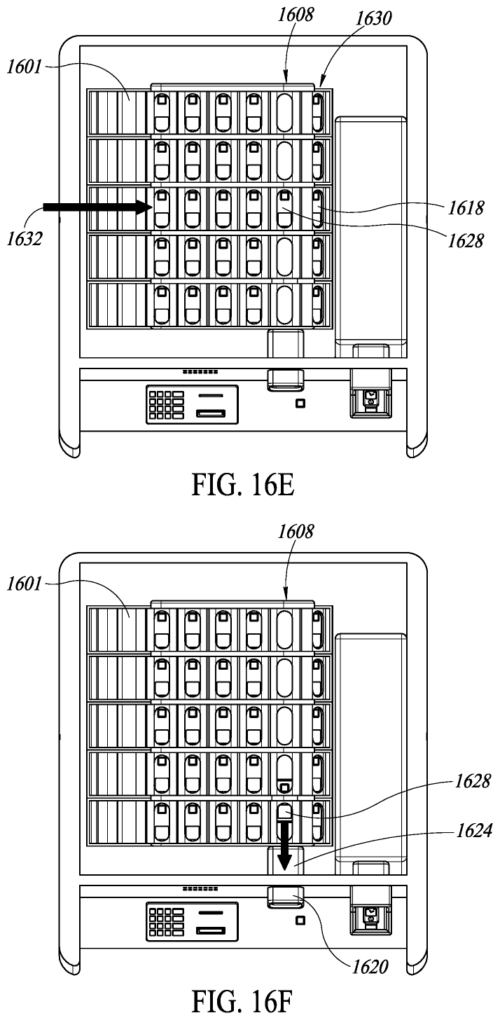

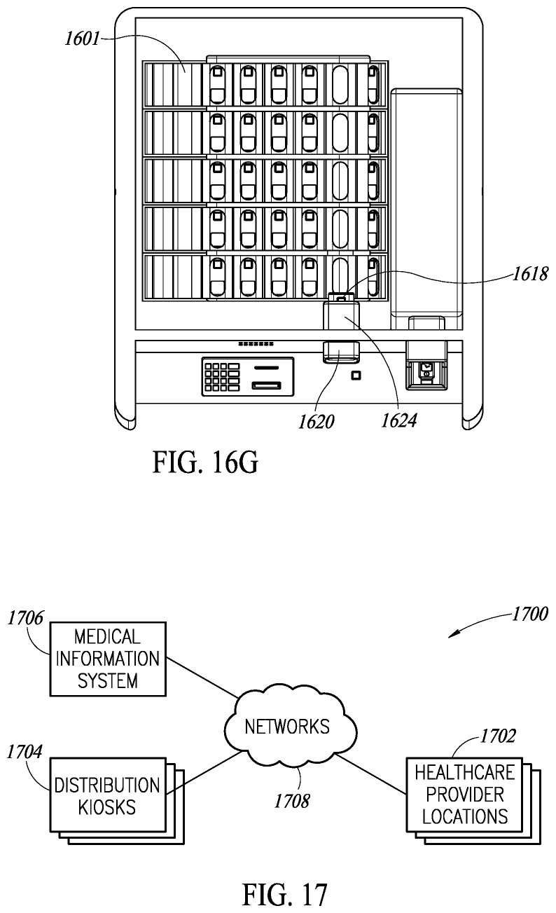

FIG. 13 is a cross-sectional view through line C-C of FIG. 12 and includes a top perspective view of the inside of the kiosk system. In some embodiments, the kiosk system has a device belt system 428 coupled to a belt actuator 430. The belt actuator 430 rotates the belt feeder 428 to facilitate alignment with the pharmaceutical agent delivery device exchange port 412. The belt feeder 428 is made up of a plurality of rotatable device receptacle loops 429 each having a plurality of device receptacle compartments 427, which are configured to receive at least one pharmaceutical delivery device, such as delivery device 100. In some embodiments the rotatable device receptacle loops 429 all rotate together around the belt actuator 430, see FIGS. 15A-15G. In some embodiments the rotatable device receptacle loops 429 rotate independently of each other, see FIGS. 16A-16G.

As an example, a user will access the kiosk system 400 when they are in need to exchange one of the pharmaceutical devices 100 or when they are in need of an emergency dose, i.e., the current pharmaceutical device 100 does not recognize the time as an authorized delivery time. It is noted that the device may be exchanged after all of the doses have been completely delivered to the patient or before all doses have been delivered, such as if the patient is preparing to be away from a kiosk for some period of time, knowing that the doses they currently have will not be sufficient for their entire period of time they will be away. It is possible that the patient could acquire the next device without returning the current device having remaining doses such that the current device can communicate with the new device to unlock or become active only after the dosages of the current device are completely delivered. In an alternative embodiment, the new device may be programmed before it is delivered to the patient with the appropriate time to become active and start providing a dose, to correspond with the last dose of the current device.

Another example would be to access an antagonist in the event someone was overdosing from consumption of an opioid. Another example, may be that the pharmaceutical device is malfunctioning, the batteries are too low, or the patient is sharing use data by connection their device to the kiosk. In yet another example, the patient may simply access the kiosk for telemedicine interaction for an ailment unrelated to opioid addiction.

To initiate an interaction with the kiosk, confirming the identity of the individual interacting with the kiosk is extremely important. Once an authorized patient is identified, the kiosk may initiate the following device exchange sequence, set forth in either FIGS. 15A-15G or 16A-16G. In FIG. 16A, which is a top down view with a top portion of the kiosk removed along the line B-B in FIG. 10, the belt system 1601 includes a plurality of belts 1602a-1602e. The plurality of belts are positioned around the belt actuator 430 in the interior chamber 435, which is secure from tampering. Each belt 1602a-1602e includes a plurality of device slots 1604a-1604n. Each slot is sized and shaped to receive one hand-held device. At a starting point, the kiosk includes a plurality of open or device-free slots and a plurality of full slots that house a variety of devices having different dosages for different types of treatment plans for patients known to be in the physical proximity to that kiosk.

Each slot includes an opening 1606 that is oval in this embodiment, which can be included to allow for long term storage without damage to the display and sensors on each device or to allow for an internal scanner or reading device to easily identify specific devices in a specific slot, such as during an internal inventory check. In this embodiment, each belt 1602a-1602e includes an open slot 1604a in a first column 1608. There are four additional columns 1610, 1612, 1614, and 1616 that are positioned on a flat, upper portion of the actuator 430.

The patient will insert a used device 1618 into a pharmaceutical agent delivery device exchange port 1620. In FIG. 16B, the device 1618 is moving into the interior chamber 435. This exchange port 1620 includes an exterior portion 1622 and an interior portion 1624. The device 1618 is transitioning from the interior portion 1624 into a channel 1626 formed by the plurality of aligned slots in the first column 1608. A filled new device 1628 is selected by CPU 422 as the next device to eject to this patient based on the patient's profile, including dosage and frequency. The new devices 1628 is positioned in the third belt 1602c in the second column 1610. The new device 1628 will either be programmed with the patient's profile and treatment plan while in the second column 1610 or as it moves out of the kiosk to the patient, such as in the exchange port 1620.

In one embodiment, the exchange port 1620 includes a data collector and data transmitter, either hard wired or wirelessly, to collect the data stored in the used device 1618 and the data to be added to the new device 1628. Some embodiments may require the user to activate an exchange mode on the device before placing the device in exchange port 1620. It is noted that due to individual patient needs, devices may be configured to include different dosage amounts and different total doses or strips to accommodate a variety of prescriptions for various treatment plans.

In FIG. 16C, the used device 1618 is moved along the first column 1608 of slots into a position or slot in the third belt 1602c in a direction indicated by arrow 1630. The movement may be achieved using another belt that is transverse to the belts 1602a-1602c. Alternatively, the movement may be using a magnetic system or other suitable means to move the used device 1618 into the correct slot for the exchange sequence.

In FIG. 16D, the used device 1618 is in the third belt 1602c adjacent to the new device 1628 to be distributed to the patient. In FIG. 16E, the third belt 1602c is rotated one slot, to the right in this orientation, as illustrated with arrow 1632. This rotation of the belt moves the used device 1618 into a third row 1630, which is starting to move away from the flat surface on the actuator. The rotation move the new device 1628 into the first column 1608. The belts are configured to rotate around as a complete loop. In this embodiment, the first column only includes a device in the third row 1602c.

In FIG. 16F, the new device is moved using the movement mechanism that moved the used device into position. The new device 1628 is moved towards the interior portion 1624 of the exchange port 1620. In 16G, the new device 1628 is programmed with the patient information for the device operation based on the treatment plan as transmitted to the kiosk from the medical information systems 1706 through the network 1708 in response to the authorized patient's interaction with the kiosk.

The data collection and transmission portion of the exchange port inputs biometric identification to the new device in order to be able of identifying an authorized patient. In some embodiments, the user will enter the privacy booth system 500 to access the kiosk system 400 by first inputting their biometric data or personal identification number into the user interface 508 to then perform a device exchange as described in this disclosure or interact with a health care provider. In some embodiments, the patient will also have to initiate biometric identification with the kiosk through both a retinal scanner 408, facial recognition, a fingerprint scanner 409, palm vein scan, palm print, and other biometric sensor techniques. It is noted that with the retinal scan, some embodiments will evaluate eye movement to determine if the patient may be experiencing certain opioid related systems. Another eye analysis that may be performed is measuring pupil dilation size.

In some embodiments, the display 406 will list the patient's demographic data and will provide prompts for the patient to select the type of encounter the patient is seeking. If the patient wants to initiate a telemedicine encounter the camera 410 and the speaker and microphone 405 will facilitate the patient communicating with a remote health care provider through the kiosk system. The display 406 will either display a video of the health care provider or will provide prompts for the patient to select. In some embodiments, the patient will use the keyboard 404 to interact with the prompts on the display 406. In other embodiments the patient will be able to interact with the display 406 as a touch screen.

In some embodiments, the authorized patient can dock the delivery device 100 with the docking station 418 to upload data from the delivery device 100 or to download data to the delivery device. In other embodiments the authorized patient will dock a wearable device, such as delivery device 300 with docking station 416. The docking station 416 will then initiate transfer of data form the delivery device 300 through a port 110 or through a wireless connection, such as through BLUETOOTH®.

In other embodiments, the user will request a single dose of a pharmaceutical agent. In some embodiments this will be an opioid agonist or an opioid antagonist. This single dose may be dispensed through dispensing mechanism 414. The kiosk system 400 can either provide this single dose from the patients request or from a request from the health care provider conducting the telemedicine session with the authorized patient.

In some embodiments, the authenticated patient can then place a used pharmaceutical delivery device 100 into the pharmaceutical agent delivery device exchange port 412, as depicted in FIG. 14. In some instances, the belt actuator 430 will move the used delivery device 100 in the direction of arrow 432 to an open receptacle compartment 427 in one of the rotatable device receptacle loops 429 of the belt feeder 428 as depicted in FIGS. 15A and 15B.

In some embodiments the belt actuator 430 under the control of the CPU 422 will move the used delivery device 100 to a specific empty spot in the belt feeder 428 next to a new delivery device 600 which contains the same dosage form of the pharmaceutical agent 118 loaded in the delivery device 600 as was in the used delivery device 100. The belt actuator 430 will then rotate the belt feeder 428 in the direction of arrow 602 as depicted in FIG. 15C. The belt actuator 430 will continue to rotate the belt feeder 428 in direction 602 until the new delivery device 600 is aligned with the pharmaceutical agent delivery device exchange port 412, as depicted in FIG. 15D. Unlike FIGS. 16A-16G, all of the belts are moved together in this embodiment. The belt actuator 430 will then move the new delivery device 600 in the direction of arrow 604 toward the pharmaceutical agent delivery device exchange port 412, as depicted in FIGS. 15E and 15F. The belt actuator 430 will continue move the new delivery device 600 until the new delivery device 600 is ejected from the pharmaceutical agent delivery device exchange port 412, as depicted in FIG. 15G.

In some embodiments, the kiosk system will be configured to allow for authorized personnel to access the kiosk to reload both individual doses of pharmaceutical agents, dispensing devices, and service any of the hardware contained within the kiosk. As part of accessing the internal workings of the kiosk, the authorized personnel may be required to enter identification such as, fingerprint identification, palm vein identification, palm print identification, face-recognition, retina identification, other biometric identification. In some instances, the kiosk may be reloaded with the mythology of an assembly that would rack-in and rack-out to facilitate rapid reloading of inventory. A rack-in and rack-out assembly may be operably linked to the central processing unit (CPU) and smart inventory system to allow inventory tracking, timestamping of reload and recording of specific authorized personnel reloading. The kiosk system may include a GPS tracking device to facilitate recover of the kiosk in case of theft or tampering. There may be one or more sensors, such as infrared sensors, electromagnetic sensors, weight sensors, cameras, to improve the security of the kiosk system. The kiosk may also contain temperature regulators and/or refrigerators for medication storage.

The specialized kiosk system may accept empty, or partially empty, delivery devices and refill the delivery device in the loading system or docking station within the kiosk. Alternatively, the device may be accepted by the kiosk system and replaced with a new delivery device. This loading system or docking station may include identification encryption methods to securely connect the contents to the authenticated patient. The loading system or docking station may also provide an electronic, magnetic, or some other mechanism that may securely lock and unlock the delivery device to facilitate reloading with a pharmaceutical agent. This loading system may include identification encryption methods to securely connect the device, cartridge, and its contents to the identified authorized patient. The loading device or docking station may include barcodes and scanning devices.

In another example of a method of using the kiosk system 400, the kiosk software, and in particular the AD software begins the interaction by recognizing the patient through biometrics. This may be completed using a combination of biometric factors, such as, but not limited to, palm vein scanning, facial recognition, or retinal scanning.

As the patient approaches the patient facing side 401 of the kiosk, AD video analytics begins recording and accessing their behavior in real time through the scanner 408. This assessment continues throughout the patient-kiosk interaction. The patient's typical mannerisms, ingestion sequence, visit time, speech, pupil size, appearance, etc., may be collected, organized and trended. The data may be used to set up a patient portfolio—which may consist of baseline appearance and behavior, as well as the typical patient-kiosk interaction, and may be automatically updated at each visit and trended over time for comparison in real-time at future kiosk visits. If the patient's interaction varies significantly from earlier interactions, the AD analytics will be able to detect the changes in behavior, send real-time alerts to the provider, and halt dispensing of the delivery device or a pharmaceutical agent.

For example, if a patient has slurred speech, excessive drowsiness, pinpoint pupils, or is slower than typical to react to kiosk prompts, the patient may be intoxicated or impaired. The ability of AD to collect data and trend cumulative visits elevates the precision for AD analytics to detect and react in real-time to a potential problem. AD may prompt the patient with further questions to confirm impairment. Once detected, the AD software may prevent dispensing by the kiosk system 400 and automatically connect a telemedicine visit with the remote provider who can assess the patient and authorize or decline dispensing.

The patient's collected biometric data will be compared to the data stored on the cloud, such as the patient's identification by, for example, the facial recognition. If the data matches, the software logs the patient into their account and the kiosk's display screen 406 displays the patient's dashboard. A visit may be as simple as requesting the daily dose of a pharmaceutical agent or it may be more complex such as reporting new medications, interacting with a health care provider at an OTP (through a microphone and speaker 405), or starting a telemedicine consult if immediate attention is warranted. Prior to dispensing a device or pharmaceutical agent, the AD will ask a series of routine questions.

The patient's personal contact information, biometrics, prescription details, primary OTP provider, AD behavior trends, telemedicine session recordings, drug testing schedule, etc., may all be updated in real-time and stored securely on the database cloud. This data may be available for review by the patient's health care provider or OTP practitioner. There may be specific metrics compiled from this data to trigger practitioner alerts such as the patient has not logged into the kiosk for their dose multiple days in a row, or the patient's behavior according to AD trending detects impairment. The cloud will also be able to generate reportable metrics such as adherence, treatment retention, diversion attempts, doses dispensed, etc.

The AD may also lock the kiosk to prevent dispensing of the device or pharmaceutical agent in certain situations. For example, if a patient had not been to a kiosk in several days, no dose would be dispensed and there would need to be a telemedicine visit with a provider to receive a new prescription. If a patient missed several days of doses, an absence protocol rule with reduced doses may be initiated with close follow up. If diversion is detected and confirmed, after the first attempt, the patient may lose privileges at the kiosk for a set number of days and must re-establish a care plan with a provider.

If a patient misses a daily dose, the kiosk system 400 may automatically send an alert, such as a text message, to the patient reminding them that they are due for a dose. The patient may be able to request alerts such as text reminders for all doses and appointments. The patient's interaction with the kiosk system 400 may be virtually touch free using voice command through the microphone and speaker 405. The display 406 may be a touch screen, as well. The patient's voice or touch-screen responses will guide the AD software in the kiosk. To decrease the patient-kiosk interaction time, the kiosk may learn the behaviors and favorite selections of each patient from each interaction with the kiosk system 400.

Once the requirements have been met and the background AD algorithm has verified that it is appropriate to dispense, the software will send instructions to the video and infrared cameras for the AD diversion detection system and to the specific medication dispensing mechanism. In some instances the kiosk system 400 will dispense a single dose of a pharmaceutical agent, such as a single dose of liquid methadone or a buprenorphine strip. The kiosk system 400 may also, for example, dispense take-home doses, the injectable long-acting buprenorphine, SUBLOCADE® and BRIXADI®, as well as naltrexone and naloxone.

In some instances the AD software in the kiosk system 400 will instruct the patient to perform a series of specific tasks on the display screen or to answer a series of questions to assess the patient's cognitive function. The patient answers will be tracked based on speed and accuracy provided and will be compared to previous patient interactions with the kiosk system 400. These specific tasks and questions may include auditory queues, visual queues, or written questions. The cognitive assessment may be another method of determining if the patient is impaired at the time of the interaction with the kiosk system 400. In some instances gamification assessment, such as finger tracing a spiral path on the display screen, may be used to assess the patient's cognitive function. The goal is to replicate procedures at methadone clinics, i.e. detect an impairment status of the patient.

If the prescription is buprenorphine, the dispensing mechanism 414 will include a strip dispenser with a series of optical sensors. The buprenorphine medication is a sublingual strip enclosed in a foil package measuring approximately 1.75″ wide by 3.5″ long and approximately 0.010″ thick. In some instances the single dose distribution subsystem 420 may include a cartridge containing a plurality of single dose strips that feeds onto an electric driver housed within the single dose distribution subsystem 420. The electric driver will advance one strip to the exterior of the kiosk through the dispensing mechanism 414 such that the patient can grasp it. A small amount of tension will remain on the strip. The patient will overcome this tension easily as they retrieve the strip but the tension will prevent the strip from loosely falling. The dispensing mechanism 414 may also contain a plurality of optical sensors to confirm that only one strip has been advanced, that it has been advanced to the correct position, and that it has been removed by the patient. In some instances the kiosk system 400 may dispense pharmaceutical agents in the form of a tablet or pill. After the medication has been dispensed successfully, the kiosk system 400 will update the kiosk inventory in real-time, through, for example, a cloud based smart inventory monitoring system.

If the prescription is methadone, the dispensing mechanism 414 may be coupled to a single dose distribution subsystem 420 which may include a high precision pump connected to a liquid methadone reservoir, a medication cup positioner with a cup cartridge, an optical sensor, a confirmation scale, and an access door actuator. The methadone medication dispensed may be a liquid that the patient drinks. Generally, the quantity of liquid ranges from 2-20 mL per dose. To start, the medication cup positioning mechanism will retrieve a cup from the cup cartridge and position it into the dispensing port. The optical sensor will confirm that the cup is in the correct position. This confirmation will enable the pump, which may be computer-controlled, is connected to the liquid reservoir to dispense a precise dose directly into the cup. To ensure up-to-date accuracy, the quantity of medication dispensed will be retrieved from the patient's real-time prescription on their cloud-based account. Once the dose has been dispensed, a confirmation scale located below the cup placement will measure the weight of both the cup and dose. This measured value will be compared to and confirmed against an expected value for that specific amount of methadone and cup. Once confirmed, the access door will be actuated to allow the patient to have access to the medication. The optical sensor will confirm that the medication has been removed by the patient and will trigger the access door closed. After the medication has been dispensed successfully, the kiosk inventory monitoring system will be updated in real time. Next, the kiosk system 400 will activate the AD dose ingestion and diversion detection system. This detection system will be an array of both video and infrared thermal cameras, such as through cameras 410 that will record the patient's actions with the dispensed medication. The patient's actions during ingestion will be analyzed in real-time by two different systems within the AD dose ingestion and diversion detection system platform: an infrared (IR) thermal image system and the continuous AD video analytics system.

In some instances, there may be a multitude of IR thermal image sensors and video sensors arranged at different angles and distances from the patient. The information collected from any combination of these IR thermal image sensors and video sensors may be utilized in conjunction with software. This software may use a combination of IR thermal image and shape tracking analysis to establish and track specific targets, such as a cooled pharmaceutical agent. This software may also analyze information from various known angles and distances to develop a virtual three-dimensional boundary box in free space, such as between a patient and a kiosk system. This software may use a virtual boundary box to generate alerts if identified targets, such as a cooled pharmaceutical agent, meet certain criteria; for example, target moves outside the designated boundary box or target diverges into multiple targets. This software may be used in conjunction with facial recognition software to identify a proximity zone, such as a patient's mouth; subsequently allowing the software to follow different alert criteria when a target and a proximity zone converge. Any combination of these IR thermal image sensors and video sensors in combination with software may be used to track the medication to the patient's mouth to determine dose ingestion or diversion.

The kiosk system 400 may include a dose ingestion and diversion detection system platform using an IR thermal image system. For this system, the medication may be stored at a cooler temperature (i.e., 59° F.) inside the kiosk. This cooler temperature may establish a thermal differential between the dispensed medication and other objects in the camera's 410 view. This thermal differential may allow the IR thermal image camera system to track in real-time the medication's specific location in space. In some instances the temperature of the pharmaceutical agent may be within the range from 59° F. to 86° F. In some instances, multiple cameras at different view angles will be used. The software may analyze each streaming video feed in real-time to locate the highest concentration of target temperature (i.e., 59° F.). The software may use a predefined field in each camera feed that the target object must remain inside of. Utilizing a plurality of camera feeds, a virtual three-dimension boundary box may be created between the patient facing side 401 of the kiosk system 400, the privacy shields 407, and the patient, creating a virtual 3-D box, in which the medication must be maintained until it is ingested. The IR thermal image target will be adjusted to account for thermal change once outside the cooled kiosk environment. Shape tracking software may be used in conjunction with IR thermal image analysis software to track objects with lower thermal capacity. For example, buprenorphine strips have a lower thermal capacity than liquid methadone.

The specific patient's ingestion location (mouth) may be determined using the facial recognition software. This software may specifically identify where the patient's mouth is located in real-time. The AD software may confirm that the tracked thermal target and the identified ingest location converge. Part of the patient's required procedure may be to open their mouth after ingestion to confirm ingestion. The IR system may then analyze the patient's open mouth to confirm that the tracked thermal target is no longer present.

The kiosk system 400 may also include a second dose ingestion and diversion detection system platform through the use of the continuous AD video analytics. This system records and analyzes the patient's appearance and actions in real-time. The use of multiple camera feeds may be used to assess the patient's typical mannerisms and ingestion sequence—which will be stored and trended with each visit. After several visits, the video analytics will develop an expected patient ingestion sequence that is unique to that patient. The AD analytics software may compare the patient's current behavior to that of the trended behaviors as well as that of the mandatory ingestion procedure that will be displayed on the kiosk display screen.

When the AD is unable to confirm ingestion and detects possible diversion, it may send an alert and perform a number of tasks, in real-time. Automated decisioning (AD) software may either prompt the patient to perform certain actions to meet the ingestion procedure or it may start a telemedicine session immediately to review the ingestion video in real-time with a remote practitioner. For example, if the infrared thermal image system determines that the medication has moved outside the virtual 3-D box or the AD video analytics identifies that the patient is displaying abnormal behavior when compared to their trend. In some instances the remote practitioner may share and review the video of the questionable activity with the patient. If the patient conforms and the practitioner accepts the patient's actions, the session will end. If the patient does not conform or there is evidence of a diversion attempt, the practitioner may revoke the patient's access to medication at the kiosk and may require them to visit an OTP for their next dose. After the first diversion attempt, if there are subsequent attempts, the patient's prescription at the kiosk may be terminated per OTP protocol and they may be required to visit the OTP for a face-to-face visit to continue care until the kiosk privilege is regained.

If the patient selects “start a telemedicine session,” the kiosk system 400 will confirm the request and send a signal to the remote telemedicine team. The patient may be placed momentarily in the telemedicine queue. This request signal may include the kiosk location, the patient information, and a review of the questions the patient has answered so the practitioner is informed as the session starts. The telemedicine feature will function much like an online meeting, similar to ZOOM™. Using the kiosk camera 410 and microphone 405, the patient will be able to interact with the practitioner in real-time. It may, at times, be urgent that a practitioner start a session if certain criteria are met during patient questioning. For example, if the patient is now pregnant or has started a new medication that interacts with the prescribed medication, their prescription will need to be reviewed. These telemedicine sessions may be recorded and stored in the patient's account for future review. If the patient selects “leave a message for a doctor,” the kiosk may respond with “please state your message.” The patient will use the kiosk's microphone 405 to record their message. The recorded message will be transcribed and forwarded, or the recorded audio will be forwarded to the proper provider or OTP support personnel. If the patient states “schedule an appointment,” the smart kiosk will access and display on the display 406 the available appointment schedule at the desired OTP location. Once the patient books the appointment, the OTP calendar will be updated in real-time with the patient's information. The kiosk then logs this appointment as a precheck reminder.

If the patient selects “get emergency help,” the smart kiosk could do one of two actions. It could start a telemedicine session with an “emergency” request signal, generating priority over other requests or, at the patient's request, the kiosk could call the local emergency services in which case the patient could use the kiosk's microphone and speaker 405 like a phone during this call. If the patient selects “update my personal information,” the patient's account information will be made accessible. This information will be secure and updated in real-time on the database cloud.

If the patient selects “log out,” the patient is logged out of their user account. The AD video analytics trending and recording the patient will remain operational until the patient completely exits. If the patient fails to log out before they exit, the AD video analytics may recognize that the patient has left and may then automatically log the patient out.

To ensure that all patient prescriptions can be readily dispensed at any location, the kiosk system 400 may maintain an inventory management system, such as a cloud-based smart inventory monitoring system. This monitoring system will ensure that the kiosk does not run out of medication, and also that the medication to be dispensed is not expired. It will also allow trending and reporting of medication used at each kiosk. With this trending, medication inventory in the kiosks will be adjusted appropriately for each specific location. This will align the costs associated with medication inventory to the actual smart kiosk patient utilization.

Embodiments of the present disclosure include devices that can deliver packaged or unpackaged strips of opioid treatment transmucosal medications, tablets, diskettes, or liquid opioid treatment medications such as methadone. The devices can provide alerts that a dose is due with vibration, with a visual notification, or with sound, among other things. The devices may interact with patients daily regarding adherence and cravings to assess risk of relapse and need for rescue, more immediate treatment, dose changes, or dose adjustment, for example. The devices may be paired or otherwise communicatively coupled with a digital therapeutic release device integrate within the hand-held or wrist-based wearable watch device where the therapeutic release device is configured to release a single naloxone treatment in case of overdose. If an overdose is detected or other unsafe vitals for the patient are detected, the device can alert or notify a contact or emergency medical services.

Kiosks of the present disclosure may have a “smart inventory” system that tracks the status of all of the devices in the kiosk and communicate real-time inventory to party responsible for refilling the kiosk through the network or cloud. The kiosk can have voice interaction with patient.

Buprenorphine may be dispensed in a variety of forms, such as a transmucosal strip or a long-acting injectable form. Methadone may be dispensed in a variety of forms, such as liquid or tablets/diskettes. Naloxone may be dispensed in a variety of forms, such as the injectable, intranasal, or within a digital therapeutic device that can auto administer naloxone in case of an overdose.

An alert such as a vibration, visual alert on screen, or ringtone can notify the patient that the dose is due. The device will also check in with patient daily to get a daily craving score to assess risk for relapse. The patient can also message using chat bot or another technology; social service referrals may be made depending on individual needs. The device can locate the nearest kiosk for a refill or exchange. The device can also interact via chat to order an exchange device through the specialty mail order pharmacy.

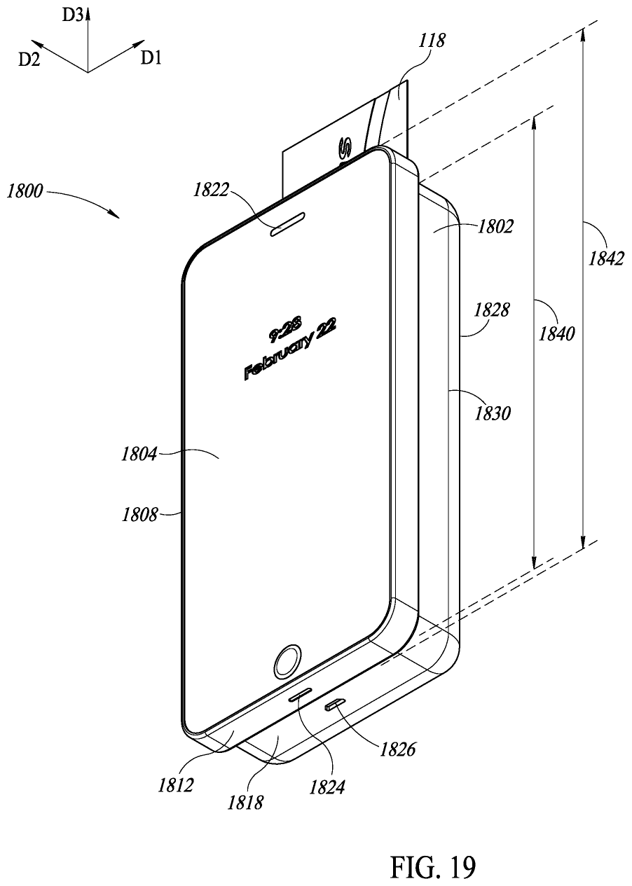

FIG. 18 is a perspective view of an alternative embodiment of the pharmaceutical agent delivery device 1800. The delivery device 1800 has an outer shell 1802 that is configured to attach a first side (not depicted) of the outer shell 1802 to a cellphone 1804. In some embodiments, the outer shell 1802 is configured to attach to a pager, a personal computing device, a tablet, or a wallet. In some embodiments the outer shell 1802 is configured to fixedly attach the first side (not depicted) of the outer shell 1802 to the second side 1806 of the cellphone 1804 which is opposite the first side 1808 of the cellphone 1804. The cellphone first side 1808 has the cellphone display monitor 1814, which may be a touch screen or other display with our without a physical keyboard. The outer shell 1802 may be attached to the cellphone 1804 so the first end 1816 of the outer shell 1802 is attached to align with the first end 1810 of the cellphone 1804. In this embodiment the second end 1818 of the outer shell 1802 is aligned with the second end 1812 of the cellphone 1804, as depicted in FIG. 19. In some embodiments the first side 1808 of the cellphone 1804 may also have a first opening 1820 and a second opening 1822. The cellphone 1804 may also have an internal microphone, not depicted.

The first opening 1820 may be aligned with a biometric sensor 1832 similar to those found in some cellphones with a fingerprint sensor. In some embodiments the first opening 1820 would also function as a button interface for the cellphone 1804. The second opening 1822 may be aligned with a speaker (not depicted) to allow the user to receive phone calls. The cellphone 1804 may be programed with an application linked via wireless transmission means, such as BLUETOOTH®, WiFi, or cellular networks with the delivery device 1800 so the biometric sensor 1832 of the cellphone 1804 would authenticate the specific user associated with the device to help ensure the pharmaceutical agent 118 is dispensed to the authenticated user. Alternatively, instead of a button or sensor 1832, the cellphone, or handheld computing device, will have a camera on the first side that is configured to perform some biometric identification, such as facial recognition.

The display 1814 on the front side 1808 of the cellphone 1804 may be a touch screen or other electronic display to provide information and interact with the patient or user. For example, the display 1814 may allow the user to interact with the application linked via wireless transmission means with the device 1800 to provide dosing, frequency, and biographical information of an authorized patient when the delivery device 1800 is associated with the authorized user. The display 1814 may provide information about battery life remaining and provide time and date information, including availability of the next dose according to the prescribed frequency.

The second side 1828 of the outer shell 1802 is opposite the first side (not depicted) of the outer shell 1802. In some embodiments the second side 1828 of the outer shell 1802 is configured to attach to the outer shell 1802 by a hinge (not depicted) such that the second side 1828 of the outer shell 1802 opens like a clam shell. Secure locking features would be incorporated to minimize tampering with the delivery device 1800 when in a closed configuration. In some embodiments, a chemical or other self-destruction system may be included to make the pharmaceutical agent inert in the event of tampering. In an alternative embodiment the second side 1828 of the outer shell 1802 would slide off the outer shell along groove 1830. In some embodiments the delivery device 1800 may be a case for the cellphone 1804. In this embodiment the cellphone 1804 would be housed within the delivery device 1800. As depicted in FIG. 19, the second end 1812 of the cellphone 1804 includes a third opening 1824 which may be a data charging port that is operably linked to the internal mechanisms of the cellphone 1804.

As depicted in FIG. 18, the first end 1816 of the outer shell 1802 has a first dimension 1832 1831 in a first direction D1. The outer shell 1802 has a second dimension 1834 in a second direction D2 that runs between the first side (not depicted) of the outer shell 1802 and the second side 1828 of the outer shell 1802. The first end 1816 of the outer shell 1802 has an opening 1836 that is configured to deliver a pharmaceutical agent 118. The third dimension 1838 in a first direction D1 is the length of the opening 1836 that is configured to deliver a pharmaceutical agent. In some embodiments the third dimension 1838 is smaller than the first dimension 1831.

As depicted in FIG. 19, the outer shell 1802 has a fourth dimension 1840 in a third direction D3 between the first end 1816 of the outer shell 1802 and the second end 1818 of the outer shell. In some embodiments the length of the fourth dimension 1840 in some embodiments is smaller than the fifth dimension 1842 in a third direction D3 that is the dimension between the first end 1810 of the cellphone 1804 and the second end 1812 of the cellphone 1804.

In some embodiments the pharmaceutical agent 118 is an individually wrapped or unwrapped medication, such as SUBOXONE®. The second end 1818 of the outer shell 1802 includes a data and data port 1826 that is operably linked to a processor, and internal mechanisms of the delivery device 1800 similarly to the devices depicted in FIGS. 1 and 7A-7C and described above. In some embodiments, the data port 1826 may be a USB-C port. The data port 1826 enables an authorized user to download or upload to and from the delivery device 1800 using encryption techniques, as described above with relation to, for example, the embodiments depicted in FIGS. 1 and 7A. In some embodiments, data is transferred to the delivery device by wireless data transfer, such as through BLUETOOTH®.

In alternative embodiments, the delivery device may be coupled to a cell phone or hand held computing device by a case or other protective packaging that is around one or more corners of the hand held computing device. The case could have a first side that receives the cell phone and securely holds the cellphone such that all or most of the corners are covered by the case. The delivery device is coupled to a second side of the case in a permanent or removable manner. The case and delivery device can be easily removed from the cell phone and to be reset or reloaded with a pharmaceutical agent. The delivery device includes at least the internal strip delivery tools. The delivery device may include all or some of the biometric identification aspects of the devices described above.

Alternatively, the delivery device can communicate wirelessly with the cell phone an all biometric identification for the delivery of the pharmaceutical agent is handled through the cell phone. The cell phone would track either within a processor in the cellphone or in the cloud, such as through an application or program loaded on the cell phone, timing for delivery of the next dost. The cell phone, with the integrated sensors, cameras, and other audio and video sensors, can evaluate a patient's current condition and build a historical behavior database over time.

What is claimed is:

1. A device, comprising:
a chassis that includes:
an outer shell;
an inner compartment within the outer shell;
a first end opposite a second end; and
a main body disposed between the first end and the second end, the main body includes:
a first side opposite a second side, the first side of the main body has a first opening and a second opening;
a biometric sensor aligned with the first opening;
a display aligned with the second opening;
a pharmaceutical agent delivery opening positioned at the first end;
a motor in the inner compartment; and
a roller coupled to the motor, the roller has a plurality of engagement loops.

2. The device of claim 1, wherein the second end of the chassis has a data port.

3. The device of claim 1, wherein the plurality of engagement loops are radial.

4. The device of claim 1, wherein the plurality of engagement loops are in contact with a pharmaceutical agent.

5. The device of claim 4, further comprising a compression device that applies pressure to the pharmaceutical agent against the engagement loops.

6. The device of claim 1, further comprising a rotation sensor configured to detect the rotational position of the roller.

7. The device of claim 1, further comprising:
a memory unit;
a processing unit operably linked to the data port, the biometric sensor, the memory unit, the motor, and the rotation sensor; and
a battery powering the processing unit, the motor, the display, the rotation sensor, and the biometric sensor.

8. The device of claim 1, further comprising a reservoir containing a substance that is released if the chassis is tampered with, wherein the substance renders the pharmaceutical agent inert.

9. A kiosk system, comprising:
a frame that includes:
an outer shell;
a plurality of inner compartments within the outer shell;
a front side opposite a back side;
the front side includes:
a display;
a camera;
a biometric sensor;
a plurality of ports;
and a user interface;
wherein at least one of the inner compartment contains a belt and a belt actuator.

10. The kiosk system of claim 9, wherein the plurality of ports comprises at least a dispensing mechanism configured to provide a single dose of a pharmaceutical agent.

11. The kiosk system of claim 10, wherein the plurality of ports comprises a pharmaceutical agent delivery device exchange interface.

12. The kiosk system of claim 9, wherein the plurality of inner compartments comprises at least a first compartment and a second compartment, where the first compartment includes a single dose mechanism operably linked to the dispensing mechanism.

13. The kiosk system of claim 12, wherein the first compartment comprises:
a processing unit;
a memory unit; and
a networking unit;
wherein the processing unit is linked to the display, the camera, the biometric sensor, the keypad, the memory, the networking unit, and the device delivery system.

14. The kiosk system of claim 12, wherein the second compartment comprises a device delivery system, the device delivery system further comprises:
a belt coupled to a belt actuator;
wherein the belt is operably coupled to a device exchange interface.

15. The kiosk system of claim 14, wherein the belt is configured to receive a pharmaceutical delivery device inserted into the device exchange interface; and wherein the belt is configured to deliver a second pharmaceutical delivery device from the second compartment out of the frame through the device exchange interface.

16. The kiosk system of claim 15, wherein the belt includes a plurality of sections, each configured to receive at least one pharmaceutical agent delivery device.

17. The kiosk system of claim 15, wherein the belt includes a plurality of rotatable device receptacle loops, wherein each loop includes a plurality of device receptacle compartments.

18. A method of a user using a kiosk system, comprising:
authenticating the user as an authorized patient with at least one biometric sensor on the kiosk system;
docking a used pharmaceutical agent delivery device on a docking station on the kiosk system;
requesting a new pharmaceutical agent delivery device from the kiosk system;
exchanging the used pharmaceutical agent delivery device with the new pharmaceutical agent delivery device with a pharmaceutical agent delivery device exchange port;
receiving the new pharmaceutical agent delivery device from the kiosk system.

19. The method of claim 18, further comprising a belt feeder including:
rotating the belt feeder with a belt actuator;
aligning the belt feeder with the pharmaceutical agent delivery device exchange port;
wherein the belt feeder includes a plurality of rotatable device receptacle loops, including a plurality sections configured to hold a pharmaceutical agent delivery device.

20. The method of claim 18, further comprising the user requesting from the kiosk system or being prescribed by the kiosk system a single dose of a pharmaceutical agent, where the pharmaceutical agent may be injectable, a strip, a film, or an oral formulation.
